(19) United States
(12) Patent Application Publication
(10) Pub. No.: US 2024/0041388 A1
(43) Pub. Date: Feb. 8, 2024

(54) SYSTEM FOR FACILITATING DIFFERENTIATION BETWEEN PARATHYROID TISSUE AND THYROID TISSUE AND APPLICATIONS OF SAME

(71) Applicant: VANDERBILT UNIVERSITY, Nashville, TN (US)

(72) Inventors: Anita Mahadevan-Jansen, Nashville, TN (US); Lisa White, Nashville, TN (US); John Phay, Bexley, OH (US); Constantine A. Paras, Nashville, TN (US); Elizabeth Kanter Bartz, Southlake, TX (US); Matthew D. Keller, Kirkland, WA (US); Nicole Mathews, St. Petersburg, FL (US); Jennifer Whisenant Amundson, Nashville, TN (US); Isaac J. Pence, Crestwood, KY (US)

(21) Appl. No.: 18/379,251

(22) Filed: Oct. 12, 2023

Related U.S. Application Data
(63) Continuation of application No. 17/289,323, filed on Apr. 28, 2021.
Application No. 17/289,323 is a continuation-in-part of application No. 16/471,049, filed on Jun. 19, 2019, now Pat. No. 11,589,801.
Application No. 16/471,049 is a continuation-in-part of application No. 15/391,046, filed on Dec. 27, 2016, now Pat. No. 10,258,275.
Application No. 15/391,046 is a division of application No. 13/056,469, filed on Jun. 14, 2011, now Pat. No. 9,687,190.
(60) Provisional application No. 62/754,717, filed on Nov. 2, 2018.
(60) Provisional application No. 62/540,767, filed on Aug. 3, 2017.
(60) Provisional application No. 62/440,112, filed on Dec. 29, 2016.
(60) Provisional application No. 61/137,584, filed on Jul. 30, 2008.
(60) Provisional application No. 61/137,520, filed on Jul. 30, 2008.

(51) Int. Cl. A61B 5/00; G01N 21/64; G01N 21/65; A61B 34/20

(52) U.S. Cl. CPC A61B 5/415; A61B 5/0071; A61B 5/0086; A61B 5/418; A61B 5/4227; G01N 21/6456; G01N 21/65; A61B 5/0075; A61B 34/20; A61B 5/742; G01N 2021/6439; G01N 2201/0221; A61B 2505/05; A61B 2034/2055

(57) ABSTRACT

A system facilitating differentiation between parathyroid tissue and thyroid tissue of a subject comprises a light source for illuminating tissue in a target region in the neck area of the subject; a light delivery device that is optically coupled with the light source for delivering a beam of light from the light source to the tissue; a collection device for collecting optical signals emitted from the tissue responsive to the beam of light; and a detector for detecting the intensities of the optical signals emitted from the tissue, wherein differences in the detected intensities enable a user to differentiate between parathyroid tissue and thyroid tissue.

### CROSS-REFERENCE TO RELATED PATENT APPLICATIONS

This application is a continuation application of U.S. patent application Ser. No. 17/289,323, filed Apr. 28, 2021, which is a national stage entry of PCT Patent Application Serial No. PCT/US2019/059610, filed Nov. 4, 2019, which itself claims priority to and the benefit of U.S. Provisional Patent Application Ser. No. 62/754,717, filed Nov. 2, 2018, and is a continuation-in-part application of U.S. patent application Ser. No. 16/471,049, filed Jun. 19, 2019, now U.S. Pat. No. 11,589,801, which is a national stage entry of PCT Patent Application Serial No. PCT/US2017/068907, filed Dec. 29, 2017, which itself claims priority to and the benefit of U.S. Provisional Patent Application Ser. No. 62/440,112, filed Dec. 29, 2016, and U.S. Provisional Application Ser. No. 62/540,767, filed Aug. 3, 2017, and is a continuation-in-part application of U.S. patent application Ser. No. 15/391,046, filed Dec. 27, 2016, now U.S. Pat. No. 10,258,275, which is a divisional patent application of U.S. patent application Ser. No. 13/056,469, filed Jun. 14, 2011, now U.S. Pat. No. 9,687,190, which is a national stage entry of PCT Patent Application Serial No. PCT/US2009/052304, filed Jul. 30, 2009, which itself claims priority to and the benefit of U.S. provisional patent application Ser. Nos. 61/137,520, and 61/137,584, both filed Jul. 30, 2008. Each of the above-identified applications is incorporated herein by reference in its entirety, respectively.

### STATEMENT AS TO RIGHTS UNDER FEDERALLY-SPONSORED RESEARCH

This invention was made with government support under Contract Nos. 9R42CA192243-03 and 1R01CA212147-01A1 awarded by the National Institutes of Health (NIH). The government has certain rights in the invention.

### FIELD OF THE INVENTION

The invention relates generally to optical assessments of bio-objects, and more particularly, to a system for facilitating differentiation between parathyroid tissue and thyroid tissue and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The endocrine system is a complex system of organs and glands which includes the thyroid and parathyroid. The anatomy of the neck is illustrated in FIG. 1. The thyroid gland regulates many developmental and metabolic processes. Common diseases of the thyroid include goiters, hyperthyroidism, hypothyroidism, benign and malignant nodules, and autoimmune diseases such as Graves' disease. Surgery is the most common treatment for Graves's disease, goiters, benign thyroid nodules, and thyroid cancers.

The parathyroid normally lies within the same region as the thyroid in the neck and functions to control calcium levels in the blood. The most common parathyroid disorder is primary hyperparathyroidism, in which one or more of the parathyroid glands become enlarged and hyperactive. This causes excess secretion of parathyroid hormone and a disruption in normal bone and mineral metabolism. The prevalence of primary hyperparathyroidism has been estimated at 21 cases per 100,000 person-years. In 80% of cases, primary hyperparathyroidism is caused by a single overactive parathyroid gland and surgical removal of the diseased parathyroid gland is the only definitive treatment.

Typically there are four tan parathyroid glands, each approximately 6 to 8 mm in size. They are typically positioned within the neck but can vary in location within the body and are sometimes intrathymic. Due to their small size and variability in position, the parathyroid glands are often difficult to distinguish from surrounding tissue and thyroid in the neck. The parathyroid visually resembles its surrounding tissue and this can extend surgical time during a parathyroidectomy, during which the surgeon is simply searching for the small organ. Accidental removal or damage to healthy parathyroids during parathyroid or thyroid surgery can result in serious complications such as hypocalcemia or hypoparathyroidism. Hypoparathyroidism may result from direct injury, devascularization, and/or disruption of the parathyroid glands. Statistics suggest that temporary and permanent hypoparathyroidism rates are between 4-20% during thyroid surgery. The most common complications of both parathyroid and thyroid surgery are postoperative hypocalcemia, vocal-cord paralysis, and hematoma.

The current surgical procedure for thyroid and parathyroid surgeries involves a systematic search within the neck in which the surgeon is mainly relying on visual inspection to identify target tissues. The incidence of complications occurring due to this subjective method is directly proportional to the extent of thyroidectomy and inversely proportional to the experience of the surgeon. The disadvantages to the current method include the lengthy duration of the surgery, the exploratory nature of the surgery, and the lack of sensitive and applicable preoperative and intra-operative imaging. Confirmation of removal of the diseased parathyroid relies on histopathology or post-operative diagnosis of symptoms. There is a need for reliable methods for identifying the parathyroid glands intraoperatively. Moreover, while surgical guidance systems have been developed and utilized for brain surgery and other organ surgery procedures, none is available for thyroid and parathyroid surgeries.

Therefore, a heretofore unaddressed need exists in the art to address the aforementioned deficiencies and inadequacies.

### SUMMARY OF THE INVENTION

In one aspect, the invention relates to a system for facilitating differentiation between parathyroid tissue and thyroid tissue of a subject comprising a light source for illuminating tissue in a target region in the neck area of the subject; a light delivery device that is optically coupled with the light source for delivering a beam of light from the light source to the tissue; a collection device for collecting optical signals emitted from the tissue responsive to the beam of light; and a detector for detecting the intensities of the optical signals emitted from the tissue, wherein differences in the detected intensities enable a user to differentiate between parathyroid tissue and thyroid tissue.

In one embodiment, the beam of light has a wavelength in the near-infrared range.

In one embodiment, the collection device is configured to collect optical auto-fluorescence signals.

In one embodiment, the system further comprises a display in communication with the detector for displaying the intensities of the optical signals emitted from the tissue.

In one embodiment, the system further comprises a filter for filtering excess ambient light to ensure desirable optical signals to be detected.

In one embodiment, the light source is a near-infrared light source.

In one embodiment, the collection device is configured to collect optical auto-fluorescence signals.

In one embodiment, the system further comprises a display in communication with the detector for displaying the intensities of the optical signals emitted from the tissue. These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1 shows a general view of the anatomy of human thyroid/parathyroid glands.

FIG. 2 schematically shows an excitation system that is utilized in one embodiment of the invention.

FIG. 3A schematically shows a system according to another embodiment of the invention.

FIG. 3B is a photo of a system reduced to practice, which is corresponding to a system according to the embodiment of the invention as shown in FIG. 4A.

FIG. 3C schematically shows a system according to yet another embodiment of the invention.

FIG. 3D is a photo of a system reduced to practice, which is corresponding to the embodiment of the invention as shown in FIG. 4C.

FIG. 3E schematically shows a system according to a further embodiment of the invention.

FIG. 3F is a photo of a system reduced to practice, which is corresponding to a system according to the embodiment of the invention as shown in FIG. 3E.

FIG. 3G schematically shows a system according to yet a further embodiment of the invention.

FIG. 4 shows an image of tissue taken with a viewer/camera coupled system according to one embodiment of the invention.

FIG. 5A shows near-infrared spectra obtained through a method according to one embodiment of the invention, where the signal from each of the parathyroid, thyroid, fat, muscle, and trachea tissues are each represented by different respective dashed line pattern.

FIG. 5B shows near-infrared spectra in the form of a normalized signal obtained through a method according to one embodiment of the invention from each of parathyroid, thyroid fat, muscle, and trachea tissues and each represented by a different respective dashed line pattern.

FIG. 6A shows average peak intensities of signals arising from parathyroid and thyroid measurements across 21 patients, as recorded by a near-infrared system according to one embodiment of the invention; as shown, parathyroid tissue consistently exhibits a stronger signal than thyroid tissue.

FIG. 6B shows normalized peak intensities of signals arising from parathyroid and thyroid measurements across 21 patients; as recorded by a near-infrared system according to ne embodiment of the invention; each measurement is normalized to the average peak of all thyroid measurements.

FIG. 7 shows in vitro auto-fluorescence of parathyroid and thyroid samples obtained from pathology all of which obtained through a method according to one embodiment of the invention; as shown, the parathyroid (PT) located on the right exhibits at least two times stronger overall auto-fluorescence than the thyroid (T) located on the left.

FIG. 8 is a photo of a clinical Raman system reduced to practice, which is corresponding to a system according to the embodiment of the invention shown in FIG. 3G; and

FIG. 9 shows processed Raman spectra of parathyroid tissue.

FIG. 10 shows schematically a laser speckle contrast imaging (LSCI) device for intraoperative assessment of parathyroid gland according to one embodiment of the invention.

FIG. 11 shows an LSCI device, according to one embodiment of the invention, where (panel a) a picture of the device, and (panel b) a schematic of an imaging end (panel b) of the device.

FIG. 12 shows speckle contrast images of a microfluidic flow phantom (panel a) and average line profile perpendicular to flow channel for both lighting conditions (panel b); white light image of surgical field with OR lights on (panel c); speckle contrast image with OR lights on (panel d); speckle contrast image with OR lights off (panel e); mean and standard deviation of speckle contrast within parathyroid for both cases (panel f). Parathyroid glands are indicated with white ellipses and the flow phantom region of interest is indicated with a white rectangle.

FIG. 13 shows images for two examples, each of vascularized (panels a-d) and compromised (panels e-h) parathyroid glands. Left column (panels a, c, e, g) shows white light images, with the corresponding speckle contrast images (panels b, d, f, h) in the right column.

FIG. 14 shows an average parathyroid gland speckle contrast grouped according to surgeon's assessment. Speckle contrast is significantly lower (p<0.0001) for vascularized glands (panel a). Of the compromised glands only 5 required transplantation, however no significant difference was observed between this sub-group and the compromised glands that were left in place (panel b). Classifying parathyroid glands based on average speckle contrast generated an ROC curve with an area under the curve of 0.935, and an optimum point with sensitivity and specificity of 92.6% and 90.6% respectively (panel c).

FIG. 15 shows changes in speckle contrast occurring seconds after blood supply ligation. A very noticeable change in speckle contrast (panels b, d) was observed in the same parathyroid gland seconds after tying off blood supply to the gland, however no such change was observed in white light images (panels a, c). Images (panel a) and (panel b) were taken before, while images (panel c) and (panel d) were taken about 30 seconds after ligation of the blood supply.

FIG. 16 shows LSCI detects vascular compromise in healthy parathyroid gland before it is visually apparent. White light image of a parathyroid gland initially thought well vascularized (panel a) did not agree with speckle contrast image (panel b). After 15 minutes, this gland was re-evaluated as devascularized (panel c) and transplanted. Speckle contrast image (panel d) supported this assessment.

FIGS. 17A-17G shows schematically a combined auto-fluorescence and laser speckle contrast imaging (LSCI) system (alternatively, device or apparatus) for intraoperative assessment of parathyroid gland vascularity of a living subject according to one embodiment of the invention. FIG. 17A shows a view of the device in the operating room. FIG. 17B is a view of an imaging head of the system. FIGS. 17C-17D show perspective and cross-sectional views of the imaging head, respectively. FIGS. 2E-2F are two perspective views showing optical paths inside the imaging head for acquiring auto-fluorescence images and LSCI images, respectively. FIG. 17G is an optical layout of the system.

FIGS. 18A-18B show schematically a combined auto-fluorescence and laser speckle contrast imaging (LSCI) system for intraoperative assessment of parathyroid gland vascularity of a living subject according to one embodiment of the invention. FIG. 18A shows a view of the device in the operating room. FIG. 18B is an optical layout of the system, where DBS represents a dichroic beamsplitter, L1 represents a first lens, L2 represents a second lens, L3 represents a third lens, LP represents a linear polarizer, LPF represents a long-pass filter, M represents a mirror, NDF represents a neutral density filter, respectively.

FIG. 19 shows a flowchart of automated image processing according to one embodiment of the invention.

FIGS. 20A-20F shows parathyroid localization steps according to one embodiment of the invention: starting with background-subtracted fluorescence image (FIG. 20A), threshold into 3 levels ((FIG. 20B); setting a second intensity level equal to background ((FIG. 20C), then locating the dominant cluster of points ((FIG. 20D); creating edge map based on this cluster of points (FIG. 20E) and fitting an active contour model to the edge ((FIG. 20F).

FIGS. 21A-21B show processed images illustrating segmented fluorescence images of parathyroid glands according to embodiments of the invention.

FIGS. 22A-22B show an in vivo example of a fluorescence image (FIG. 22A) and probability of parathyroid devascularization (FIG. 22B) according to one embodiment of the invention.

FIG. 23 shows white light, auto-fluorescence and speckle contrast images of a diseased parathyroid gland before and after blood supply ligation, according to one embodiment of the invention. The parathyroid is more fluorescent than surrounding tissue and an increase in speckle contrast after ligation shows devascularization of the gland.

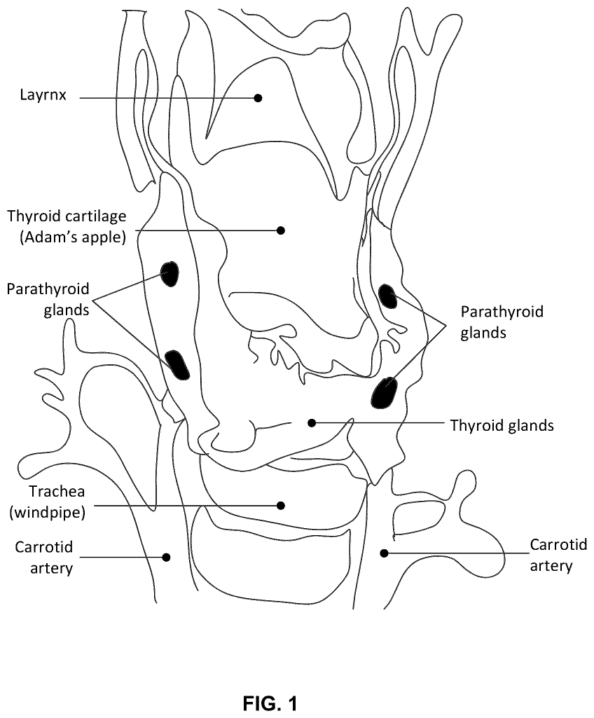

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, the term “subject” refers to a human being such as a patient, or a mammal animal such as a monkey.

As used herein, the term “Raman spectroscopy” refers to an optical technique that probes the specific molecular content of a sample by collecting inelastically scattered light. As photons propagate through a medium, they undergo both absorptive and scattering events. In absorption, the energy of the photons is completely transferred to the material, allowing either heat transfer (internal conversion) or re-emission phenomena such as fluorescence and phosphorescence to occur. Scattering, however, is normally an elastic process, in which the incident photons retain their energy. In Raman scattering, the photons either donate or acquire energy from the medium, on a molecular level making it an inelastic process. In contrast to fluorescence, where the energy transfers are on the order of the electronic bandgaps, the energy transfers associated with Raman scattering are on the order of the vibrational modes of the molecule. These vibrational modes are molecularly specific, giving every molecule a unique Raman spectral signature.

Raman scattering is a very weak phenomena, and therefore practical measurement of Raman spectra of a medium requires high power excitation laser sources and extremely sensitive detection hardware. Even with these components, the Raman spectra from tissue are masked by the relatively intense tissue auto-fluorescence. After detection, post processing techniques are required to subtract the fluorescence background and enable accurate visualization of the Raman spectra. Raman spectra are plotted as a function of frequency shift in units of wavenumber (cm⁻¹). The region of the Raman spectra where most biological molecules have Raman peaks is from 500 to 2000 cm⁻¹. In contrast to fluorescence spectra, Raman spectra have sharp spectral features that enable easier identification of the constituent sources of spectral peaks in a complex sample. In the context of detecting the changes that cancerous tissues undergo, differences in the Raman spectral features that correlate to the increased nucleic acid content in neoplastic cells has been observed.

The term “fluorescence spectroscopy” or its synonyms “fluorometry” or “spectrofluorometry” refers to a type of electromagnetic spectroscopy which analyzes fluorescence from a sample. It involves using a beam of light, usually ultraviolet light, that excites the electrons in molecules of certain compounds and causes them to emit light of a lower energy, typically, but not necessarily, visible light. A complementary technique is absorption spectroscopy.

Fluorescence spectroscopy utilizes that fact that molecules have various states referred to as energy levels. Fluorescence spectroscopy is primarily concerned with electronic and vibrational states of energy. Generally, the sample being examined will have a ground electronic state (a low energy state) of interest, and an excited electronic state of higher energy. Within each of these electronic states are various vibrational states.

In fluorescence spectroscopy, the sample is first excited, by absorbing a photon, from its ground electronic state to one of the various vibrational states in the excited electronic state. Collisions with other molecules cause the excited molecule to lose vibrational energy until it reaches the lowest vibrational state of the excited electronic state.

The molecule then drops down to one of the various vibrational levels of the ground electronic state again, emitting a photon in the process. As molecules may drop down into any of several vibrational levels in the ground state, the emitted photons will have different energies, and thus frequencies. Therefore, by analyzing the different frequencies of light emitted in fluorescence spectroscopy, along with their relative intensities, the structure of the different vibrational levels can be determined.

In a typical measurement, the different frequencies of fluorescence light emitted by a sample are measured, holding the excitation light at a constant wavelength. This is called an emission spectrum. An excitation spectrum is measured by recording a number of emission spectra using different wavelengths of excitation light.

As used in this disclosure, the term “auto-fluorescence” refers to the fluorescence produced by a molecule of interest without the use of exogenous markers. Auto-fluorescence may serve as a useful diagnostic indicator such as in the case of “biological auto-fluorescence”, which refers to the fact that cells contain molecules, which become fluorescent when excited by UV/VIS (typically 400-700 nm) radiation of suitable wavelength. This fluorescence emission, arising from endogenous fluorophores, is an intrinsic property of cells and is called auto-fluorescence to be distinguished from fluorescence signals obtained by adding exogenous markers. The majority of cell auto-fluorescence originates from mitochondria and lysosomes. Together with aromatic amino acids and lipo-pigments, the most important endogenous fluorophores are pyridinic (NADPH) and flavin coenzymes. In tissues, the extracellular matrix often contributes to the auto-fluorescence emission more than the cellular component, because collagen and elastin have, among the endogenous fluorophores, a relatively high quantum yield. Changes occurring in the cell and tissue state during physiological and/or pathological processes result in modifications of the amount and distribution of endogenous fluorophores and chemical-physical properties of their microenvironment. Therefore, analytical techniques based on auto-fluorescence monitoring may be utilized in order to obtain information about morphological and physiological state of cells and tissues. Moreover, auto-fluorescence analysis can be performed in real time because it does not require any treatment of fixing or staining of the specimens.

As used in this disclosure, the term, “laser speckle contrast imaging” or its abbreviation “LSCI” refers to a technique for imaging flow for assessment of parathyroid gland vascularity during endocrine surgery, which utilizes intrinsic tissue contrast from dynamic light scattering and provides a relatively simple technique for visualizing detailed spatiotemporal dynamics of blood flow changes in real-time. Laser speckle is the random interference pattern produced when coherent light scatters from a random medium and can be imaged onto a detector. Motion from scattering particles, such as red blood cells in the vasculature, leads to spatial and temporal variations in the speckle pattern. Speckle contrast analysis quantifies the local spatial variance, or blurring, of the speckle pattern that results from blood flow. Areas with greater motion have more rapid intensity fluctuations and therefore have more blurring of the speckles during the camera exposure time. LSCI can be used to quantify relative changes in blood flow.

The LSCI technique analyzes the interference pattern produced when coherent light is incident on a surface. Minute differences in path length created by the light waves scattering from different regions of the surface produce bright and dark spots of constructive and destructive interference respectively, termed as a speckle pattern. This speckle pattern fluctuates depending on how fast particles are moving within a few microns of the surface. Blurring of the speckle pattern occurs when the motion is fast relative to the integration time of the detector. Analyzing this spatial blurring provides contrast between regions of faster versus slower motion and forms the basis of LSCI. This technique is sensitive to microvascular perfusion and has been employed in a variety of tissues where the vessels of interest are generally superficial, such as the retina, skin and brain. Parathyroid glands are densely packed with blood vessels, given that they secrete PTH to the entire body. Furthermore, their small size (3-8 mm) makes many of these vessels superficial, making these glands suitable targets for assessment using LSCI. Certain aspects of this invention disclose a combined auto-fluorescence and LSCI system for assessment of parathyroid gland vascularity during endocrine surgery.

As used in this disclosure, “charge-coupled device” or “CCD” refers to an analog shift register that enables the transportation of analog signals (electric charges) through successive stages (capacitors), controlled by a clock signal. Charge-coupled devices can be used as a form of memory or for delaying samples of analog signals. Today, they are most widely used in arrays of photoelectric light sensors to serialize parallel analog signals. In a CCD for capturing images, there is a photoactive region (an epitaxial layer of silicon), and a transmission region made out of a shift register (the CCD, properly speaking).

An image is projected through a lens onto the capacitor array (the photoactive region), causing each capacitor to accumulate an electric charge proportional to the light intensity at that location. A one-dimensional array, used in line-scan cameras, captures a single slice of the image, while a two-dimensional array, used in video and still cameras, captures a two-dimensional picture corresponding to the scene projected onto the focal plane of the sensor. Once the array has been exposed to the image, a control circuit causes each capacitor to transfer its contents to its neighbor (operating as a shift register). The last capacitor in the array dumps its charge into a charge amplifier, which converts the charge into a voltage. By repeating this process, the controlling circuit converts the entire semiconductor contents of the array to a sequence of voltages, which it samples, digitizes and stores in some form of memory.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Thyroid and parathyroid diseases combine the fields of endocrinology and oncology leading to a complex combination of conditions. When the disease cannot be treated by other methods, surgical means are used to remove the diseased gland(s). The thyroid gland, parathyroid glands, nerves, adipose tissue, and lymph nodes are closely positioned in the neck region. Due to their close proximity and tendency to blend into each other, many of these structures, specifically the parathyroid glands, are difficult to distinguish visually during endocrine surgery. The situation is further complicated by its small size and variability in position. Surgeons must ultimately rely on visual inspection to identify the different tissues, which can be subjective and often inconclusive. Complications occur when the parathyroid is accidentally injured or removed during thyroidectomies or only partially removed in the case of parathyroidectomies.

Existing methods for identifying parathyroid glands are limited and have deficiencies that can result in surgical complications. Current technology relies on histopathology or post-operative diagnosis of symptoms to determine if the parathyroid was accidentally or incompletely removed. An accurate, automated diagnostic method could allow faster, more effective patient management. Optical spectroscopy can detect differences in tissue architecture and biochemical composition. In particular, fluorescence spectroscopy has been of considerable interest in the development of new clinical diagnostic tools. Fluorescence measurements of human tissue can be made in real-time, without tissue removal and diagnosis based on tissue fluorescence can be easily automated. Fluorescence molecules typically exhibit signal in the UV/VIS wavelengths, or about 400-700 nm. As excitation wavelengths become longer, auto-fluorescence decreases. It is believed that for the first time, near-infrared auto-fluorescence has been utilized in the invention and attractive to the inventors due to their increased penetration depth in biological tissues.

In one aspect, the invention provides, among other things, a system facilitating differentiation between parathyroid tissue and thyroid tissue of a subject. The system comprises a light source for illuminating tissue in a target region in the neck area of the subject; a light delivery device that is optically coupled with the light source for delivering a beam of light from the light source to the tissue; a collection device for collecting optical signals emitted from the tissue responsive to the beam of light; and a detector for detecting the intensities of the optical signals emitted from the tissue, wherein differences in the detected intensities enable a user to differentiate between parathyroid tissue and thyroid tissue.

In one embodiment, the beam of light has a wavelength in the near-infrared range.

In one embodiment, the collection device is configured to collect optical auto-fluorescence signals.

In one embodiment, the system further comprises a display in communication with the detector for displaying the intensities of the optical signals emitted from the tissue.

In one embodiment, the system further comprises a filter for filtering excess ambient light to ensure desirable optical signals to be detected.

In one embodiment, the light source is a near-infrared light source.

In one embodiment, the collection device is configured to collect optical auto-fluorescence signals.

In one embodiment, the system further comprises a display in communication with the detector for displaying the intensities of the optical signals emitted from the tissue.

In another aspect, the invention provides a process or method of using near-infrared auto-fluorescence for the identification of the parathyroid in vivo during endocrine surgery. Such a method could prevent accidental or incomplete removal of parathyroid glands.

In yet another aspect, the invention provides real time imaging systems that can detect the near-infrared auto-fluorescence from the tissues in the neck in real time to guide surgical resection or surgical procedure.

In one embodiment, an imaging system was built to detect near-infrared auto-fluorescence. The system includes a near-infrared light source for emitting a beam of near-infrared light, a light guide or optical fiber(s) that delivers the light to the tissue, a camera with a filter to block unwanted wavelengths, and a computer with a controller in communication with each component/device of the system to coordinate the image capture. In operation, tissue was placed under the camera, the near-infrared auto-fluorescence from the tissue was collected and acquired through the camera, and stored in a memory for further processing.

In another embodiment, an imaging system provides an x-y image (or video image) of the field including NIR wavelengths with the sensitivity needed for tissue auto-fluorescence measurements. An exemplary device was built with a FIND-R-SCOPE®. Such a system can be made as a portable, hand-held device, in which the image is viewed through a built-in viewer by a user such as a surgeon to see the auto-fluorescence from the tissue or from the targeted area of the tissues on site. The viewer can also be connected to a display with or without a camera attachment. The imaging system can be used to guide the surgeon intra-operatively through a surgical procedure to identify the tissues that need to be acted on.

In short, according to several embodiments of the invention, near-infrared (NIR) auto-fluorescence is used to identify parathyroid glands during thyroid and parathyroidectomies. The invention utilized the discovery that parathyroid tissue had unique optical signals to identify the parathyroid glands and differentiate them from other tissues in the neck intra-operatively, in which the intensity of the parathyroid signal was compared to the auto-fluorescence of the surrounding tissue including the thyroid gland. Several fluorescence measurements were conducted according to the invention on patients who were undergone endocrine surgery at the Vanderbilt University Medical Center. In each of the measurements, the parathyroid had markedly higher levels of auto-fluorescence. These results indicate that NIR auto-fluorescence provides an excellent tool to locate parathyroid tissue in endocrine surgery. Without intent to limit the scope of the invention, exemplary methods and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Intraoperative Identification of Parathyroid Tissue and Thyroid Tissue

### Embodiment 1

This example in part illustrates a method or process, with corresponding apparatus or system, according to one embodiment of the invention.

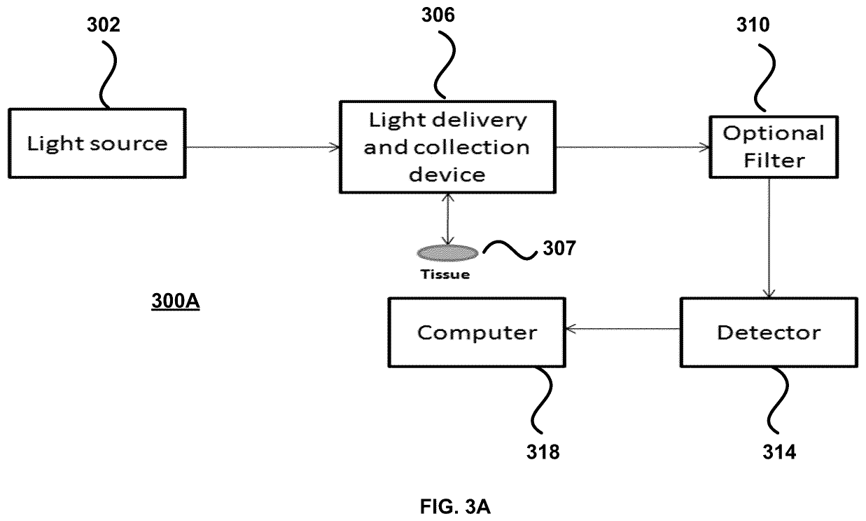

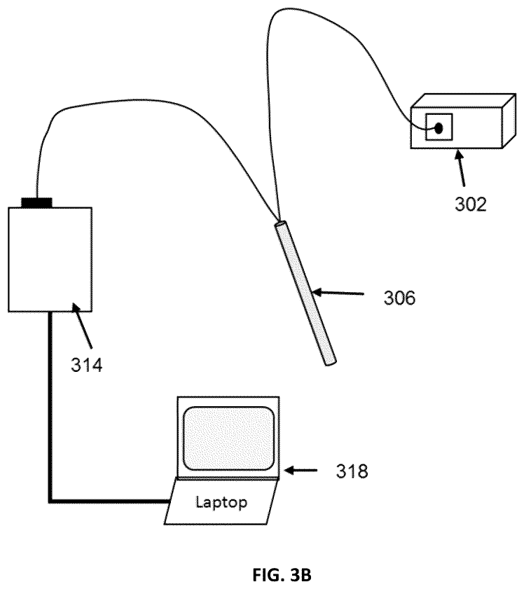

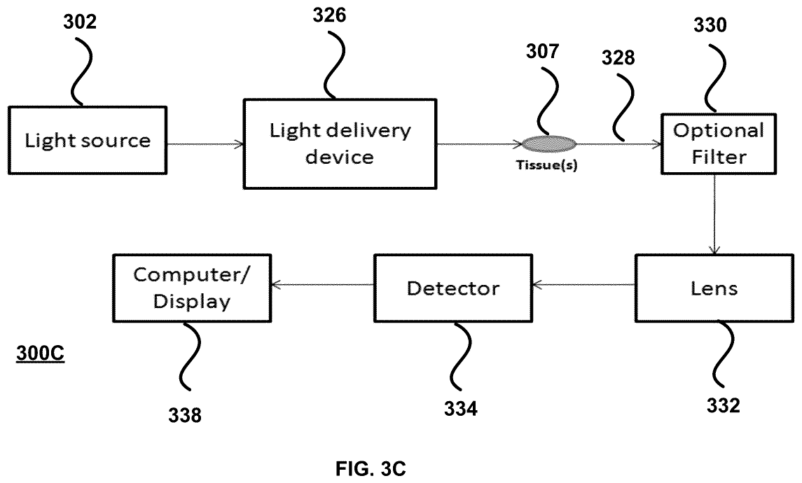

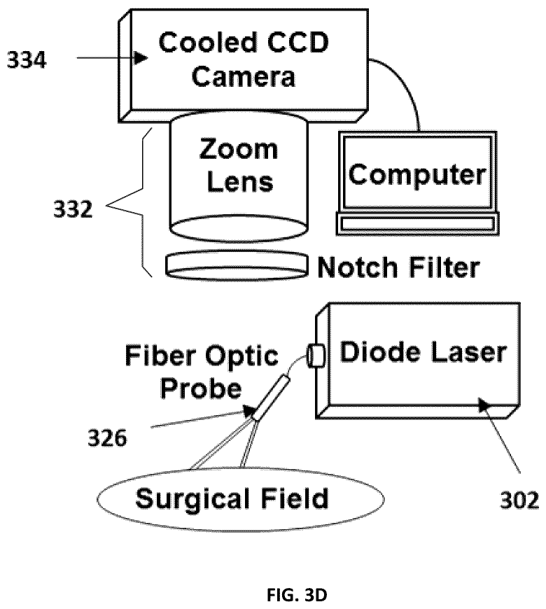

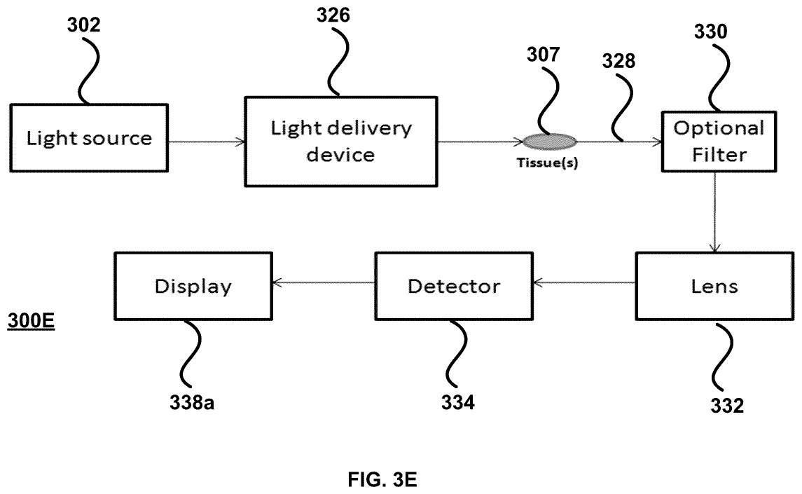

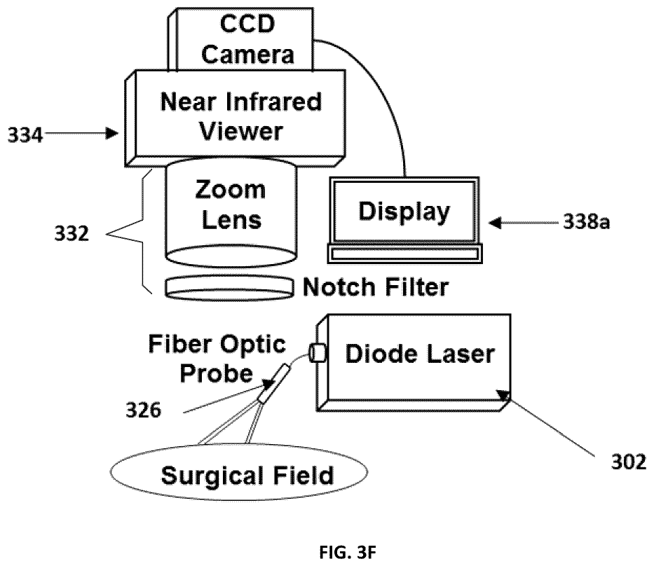

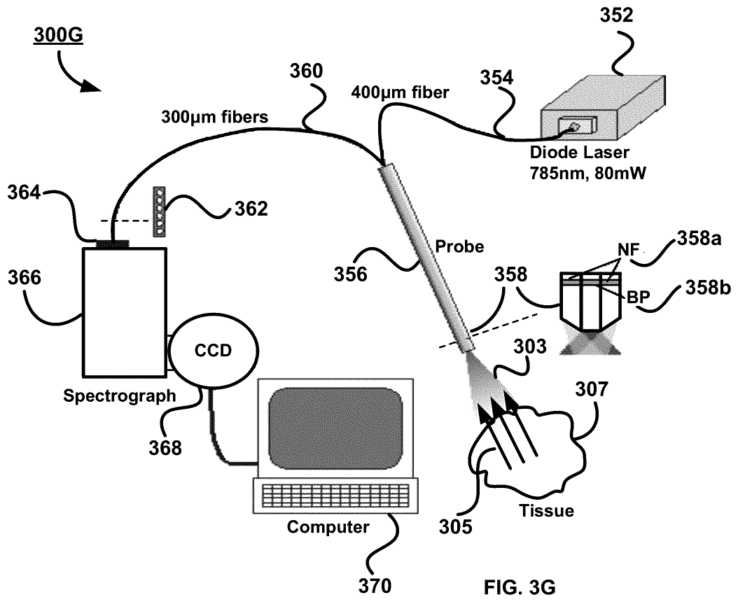

Referring now to FIG. 3E, a system 300E is schematically shown in a block diagram. The system 300E has a light source 302, which is designed to provide a beam of light with appropriate excitation wavelengths and energy so as to excite certain molecules of sample tissue 307, when receiving the beam of light, to generate auto-fluorescence emission. The system 300E also has a light delivery device 326, which is optically coupled with the light source 302 and delivers the beam of light from the light source 302 to the tissue 307. An optional filter 330 can be strategically positioned in an optical path 328 along which optical signals emitted from the tissue 307 responsive to the incident beam of light travel and utilized to reduce excess wavelengths and/or ambient light to ensure desirable optical signals to be detected. The system 300E further has a detector 334, which detects and records the optical signals emitted from the tissue 307. A lens 332 is positioned between the tissue 307 and the detector 334 to receive the optical signals emitted from the tissue and focus them on a desired spot on the detector 334. If an optional filter 330 is utilized, lens 332 will be positioned between the optional filter 330 and the detector 334. A display 338a, which has imaging processing and display capacity, is at least in communication with the detector 334, where the display 338a is adapted with appropriate controller cards and software to control signal acquisition, process data and display spectra, among other things. The display 338a displays instant images corresponding to the optical signals emitted from the tissue 307, which may guide a surgeon or a medical professional through a medical procedure to identify tissues and act on them accordingly.

FIG. 3F shows a photo of a system corresponding to system 300E of FIG. 3E, which was reduced to practice.

A system according to one embodiment of the invention allows a surgeon to correctly differentiate between thyroid and parathyroid tissue intra-operatively and immediately. Due to their differing optical properties, thyroid, parathyroid and the surrounding neck tissues can be easily differentiated intra-operatively by the system. When a 785 nm laser excites a neck area of tissue, the parathyroid fluoresces with a greater intensity than the surrounding thyroid tissue. Normal tissues other than thyroid or parathyroid in the neck region will not create detectable auto-fluorescence. Both the thyroid and parathyroid fluoresce at about 822 nm, with the parathyroid fluorescing with greater intensity. The system described in this Example allows the 822 nm light created by the thyroid and parathyroid to be viewed directly by the surgeon as visible light. The parathyroid visualization unit according to one embodiment of the invention is easy to use, requiring as little training as possible. It also effectively decreases operative time and the number of unsuccessful operations, thereby greatly reducing the need for costly secondary operations. More importantly, the system would reduce the rate of accidental removal or injury to the parathyroid. The system may be coupled with equipment already present in the operating room (“OR”).

There are currently no methods consistently used intra-operatively to differentiate between thyroid and parathyroid tissue. The system according to one embodiment of the invention has following features: it uses optical fibers running from a light source such as a 785 nm laser source to excite the tissue. The system has a display device, allowing for the surgeon to view the differing auto-fluorescence of the two or more tissue types, instead of interpreting a numerical intensity value. The system is attachable to a head-unit that the surgeon could wear during surgery.

The new system is in real-time. This real-time factor could decrease time in the OR by creating an intra-operative imaging modality. This would eliminate the need to send tissue samples to histology simply to determine tissue type during surgery and thus reduce any wait times associated with this. The system is adapted to be able to excite an area equal to the size of the surgeon's incision.

Methodology. To allow the system, according to one embodiment of the invention, to have visualization ability, several cameras and viewers were tried and/or evaluated to determine which would be most suitable for this feature. A viewer system with peak sensitivity around 825 nm and the ability to visually show the surgeon the NIR light within the visual spectrum was needed. The FIND-R-SCOPE® Infrared Viewer Model 84499A was the final choice. This viewer has spectral sensitivity from 350 to 1350 nm with peak sensitivity at 800 nm, very close to the 822 nm fluorescence coming from the parathyroid tissue. This model is also lightweight, a feature preferred for attachment to a head-unit. To view only the auto-fluorescence of the tissue, an 820 nm long pass filter was placed in front of the viewer. This was important because it removed the high intensity 785 nm laser light as well as visible light and would reduce any background signal from overhead lights during surgery.

Additionally, the different means of exciting a larger area of tissue than a laser independently would excite was tried and/or evaluated. The diameter of the laser beam was measured at approximately 0.3 cm which is rather smaller than the desired one square centimeter. A liquid light guide was utilized because it would excite the desired area of tissue while, at the same time, maintaining the power necessary to excite the tissue. A liquid light guide from Newport® (Model: 77639 Liquid Light Guide, 420-2000 nm, 0.3 in Core, 3.9 in Bend Radius, 79 in Length) was used in practice. This liquid light guide is compatible with light in the NIR, has a great enough numerical aperture such that a fiber from the laser could be directly coupled into the liquid light guide, and is long enough to allow the laser source to be separate from the excitation-detection system. Other liquid light guides having comparable parameters can also be utilized to practice the invention.

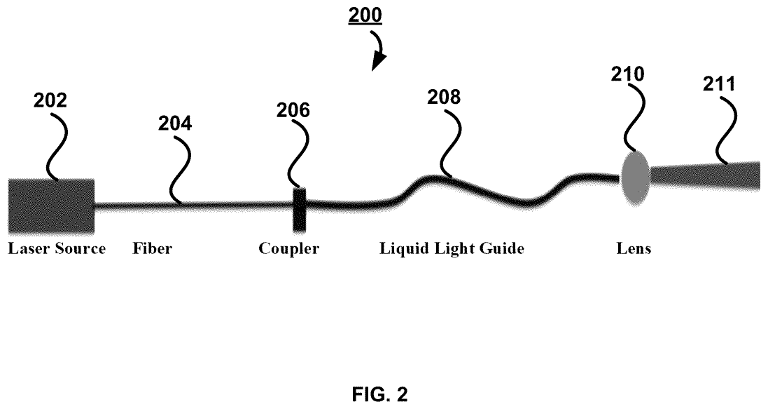

The excitation setup of the system according to one embodiment of the invention, as shown in FIG. 2, used a 785 nm laser 202 coupled to an optical fiber 204 with a diameter of 400 μm and a numerical aperture of 0.22. This cable 204 was then directly coupled to the liquid light guide 208, via coupler 206. The numerical aperture of the liquid light guide 208 is greater than that of the fiber 204 and thus no optics were needed to couple the fiber 204 to the liquid light guide 208. A Newport® lens 210 with a focal length of 25.4 mm was used at an approximate distance of 6 cm from the liquid light guide 208 to focus the beam 211. The diameter of the beam 211 when focused was approximately 1.2 cm.

A second option was investigated to couple the viewer to a camera and then view the fluorescing tissue on an external screen. A Sony® SC-75 CCD video camera module coupled with the FIND-R-SCOPE® and Pinnacle® software was used to view the fluorescing tissue on an external screen. This setup was also used to take pictures and video of the auto-fluorescence.

Tissue Fluorescence Comparison. Finally the complete system as photographed in FIG. 3F was tested. A cage was built in front of the liquid light guide that created a simple reference to ensure that the light from the liquid light guide was properly focused. This excitation system was used to excite human tissue samples of thyroid and parathyroid. The auto-fluorescence of both the thyroid and the parathyroid were viewed using the viewer and pictures were taken using the camera/viewer-coupled system.

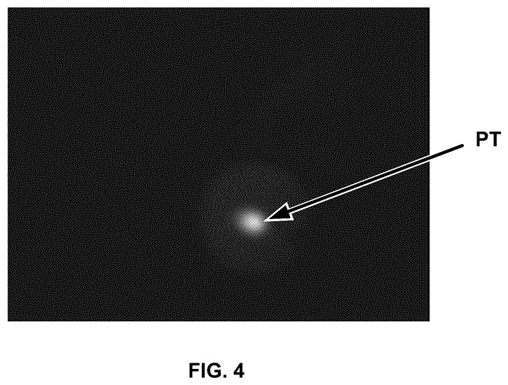

Power Needed for Minimum Detectability of Tissue Fluorescence. The minimum power at which near-infrared light could be viewed using the viewer was determined to be 3 nW. The spectral resolution of the viewer/camera-coupled system was found to be 8 nW. FIG. 4 is a picture taken with the viewer/camera-coupled system, showing parathyroid tissue (PT).

Viewing Tissue Auto-Fluorescence. The filter applied to the viewer blocked all wavelengths below 820 nm. Because the excitation laser used operates at a 785 nm, any light from the laser was blocked by the filter, and it cannot be seen. However, parathyroid auto-fluorescence, was still visible through the device. As stated previously, parathyroid tissue fluoresces at a much greater intensity than fat or thyroid tissue. The human eye cannot differentiate the parathyroid from surrounding tissue because the parathyroid fluoresces at a wavelength outside of the visible spectrum. This system would allow a surgeon to visually differentiate between tissue types based on auto-fluorescence within the NIR range.

Physical Specifications of the Device. The system's small size and mobility make it adaptable for different operating room layouts and integratable with existing surgical equipment. The fact that it can be moved easily from place to place makes it cost-effective, as it reduces the need for the purchase of multiple viewing systems to be used by a single hospital.

Thus, a system according to one embodiment of the invention as illustrated in Example 1 shows that by using the optical properties of the thyroid and parathyroid the system is able to visually differentiate the different tissue types during thyroid and parathyroid surgeries. This system when developed into a full surgical system can be installed into operating rooms. Improvements including increased spatial and spectral resolution for both the viewer and any secondary camera system, as well as a fully head mountable detector, viewer and excitation system can be made under the principle of the invention. This system will help avoid injury/removal of the parathyroid as well as reduce surgical time and the need for secondary surgeries because the system will allow the surgeon to quickly and efficiently differentiate between surrounding tissue, thyroid and parathyroid. The visual cue will help the surgeon quickly differentiate between tissue types and possibly remove the need for sending tissue samples to histology during surgery when testing for accidental removal of parathyroid tissue. This will reduce time in surgery and will ensure that the correct tissue is removed avoiding post-operative hypoparathyroidism and eliminating the need for some secondary surgeries. Other possible improvements could include image processing to create a real time color map image of the fluorescent profile.

### Embodiment 2

This example in part illustrates a method or process, with corresponding apparatus or system, according to another embodiment of the invention.

Referring now to FIG. 3C, a system 300C is schematically shown in a block diagram. The system 300C has a light source 302, which is designed to provide a beam of light with appropriate excitation wavelengths and energy so as to excite certain molecules of sample tissue 307, when receiving the beam of light, to generate auto-fluorescence emission. The system 300C also has a light delivery device 326, which is optically coupled with the light source 302 and delivers the beam of light from the light source 302 to the tissue 307. An optional filter 330 can be strategically positioned in an optical path 328 along which optical signals emitted from the tissue 307 responsive to the incident beam of light travel and utilized to reduce excess wavelengths and/or ambient light to ensure desirable optical signals to be detected. The system 300C further has a detector 333, which detects and records the optical signals emitted from the tissue 307. A lens 332 is positioned between the tissue 307 and the detector 333 to receive the optical signals emitted from the tissue and focus them on a desired spot on the detector 333. If an optional filter 330 is utilized, lens 332 will be positioned between the optional filter 330 and the detector 333. A computer 338, which has imaging display capacity or is associated with a display device, is at least in communication with the detector 333, where the computer 338 is adapted with appropriate controller cards and software to control signal acquisition, process data and display spectra, among other things. The display displays instant images processed by the computer 338 from the optical signals emitted from the tissue 307, which may guide a surgeon or a medical professional through a medical procedure to identify tissues and act on them accordingly.

FIG. 3D shows a photo of a system corresponding to system 300C of FIG. 3C, which was reduced to practice.

Imaging System and Measurements. Detection using a probe-based NIR fluorescence system provided a fast and accurate way to detect parathyroid glands. This method is particularly useful when the parathyroid is located deep in the neck and is not necessarily exposed. However, in most cases, an imaging system would provide more spatial information to the surgeon improving upon the guidance using the probe based system. An NIR imaging setup was assembled to assess the feasibility of imaging the parathyroid. Tissue was excited with the same 785 nm diode laser, defocused to provide a ˜6 cm diameter spot size. A notch filter was used to block reflected laser light. Images were obtained using a Aspherical HF 23-80 mm f/3.5-5.6 macro lens (Sigma, Ronkonkoma, NY) with a PhotonMAX 512 (Princeton Instruments, Trenton, NJ) charge-coupled device (CCD) camera. Images were recorded using Winview software (Princeton Instruments).

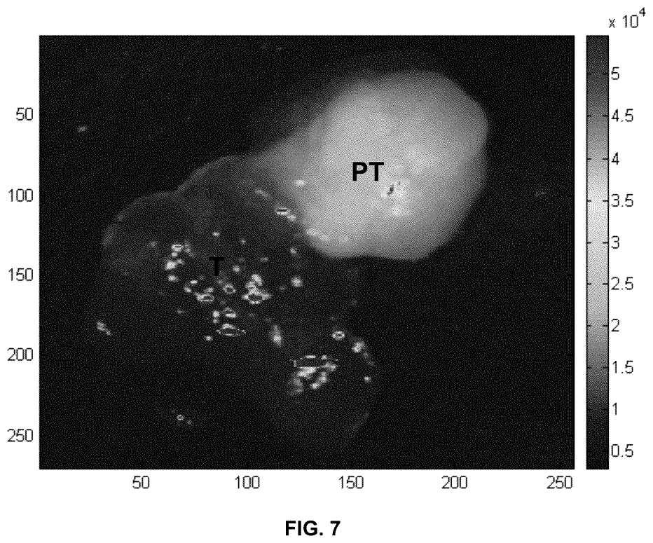

Now referring also to FIG. 7, frozen samples of thyroid and parathyroid tissue were obtained and thawed and at room temperature in phosphate-buffered saline in a petri dish. A non-reflective, non-fluorescent layer was placed between the samples and the dish. The fluorescent room lights were turned off and the diode laser was used to illuminate both tissue samples equally. The light was delivered at a slight angle such that background specular reflection from the tissue was minimized. The camera was placed 7 inches above the tissues and focused. Images were taken with a 200 ms acquisition time using Winview. The images were processed using MATLAB to remove speckle by applying a median filter. FIG. 7 shows both glands fluorescing. The parathyroid is on the right and the thyroid on the left. The parathyroid fluoresces just over twice as much as the thyroid tissue. Not only was it possible to capture the intrinsic fluorescence with a camera but the parathyroid exhibits stronger auto-fluorescence in vitro as well as in vivo.

Thus, a system according to one embodiment of the invention as illustrated in Example 2 shows that by using the optical properties of the thyroid and parathyroid the system is able to visually differentiate the different tissue types during thyroid and parathyroid surgeries. This system can be developed into a full surgical system that can be installed into operating rooms. This system will help avoid injury/removal of the parathyroid as well as reduce surgical time and the need for secondary surgeries because the system will allow the surgeon to quickly and efficiently differentiate between surrounding tissue, thyroid and parathyroid. The visual cue will help the surgeon quickly differentiate between tissue types and possibly remove the need for sending tissue samples to histology during surgery when testing for accidental removal of parathyroid tissue. This will reduce time in surgery and will ensure that the correct tissue is removed avoiding post-operative hypoparathyroidism and eliminating the need for some secondary surgeries. Other possible improvements could include image processing to create a real time color map image of the auto-fluorescence profile.

### Embodiment 3

This example in part illustrates an apparatus or system, which can be utilized to practice a method or process according to one embodiment of the invention.

Referring now to FIG. 3A, a system 300A is schematically shown in block diagram. The system 300A has a light source 302, which is designed to provide a beam of light with appropriate excitation wavelengths and energy so as to excite certain molecules of sample tissue 307, when receiving the beam of light, to generate fluorescence emission. The system 300A also has a light delivery and collection device 306, which is optically coupled with the light source 302 and delivers the beam of light from the light source 302 to the tissue 307 and collects optical signals emitted from the tissue 307 responsive to the incident beam of light. An optional filter 310 can be utilized to reduce excess wavelengths and/or ambient light to ensure desirable optical signals to be detected. The system 300A further has a detector 314, which detects and records the optical signals emitted from the tissue. A computer 318 is at least in communication with the detector 314, where the computer 318 is adapted with appropriate controller cards and software to control signal acquisition, process data and display spectra, among other things.

FIG. 3B shows a photo of a system corresponding to system 300A of FIG. 3A, which was reduced to practice.

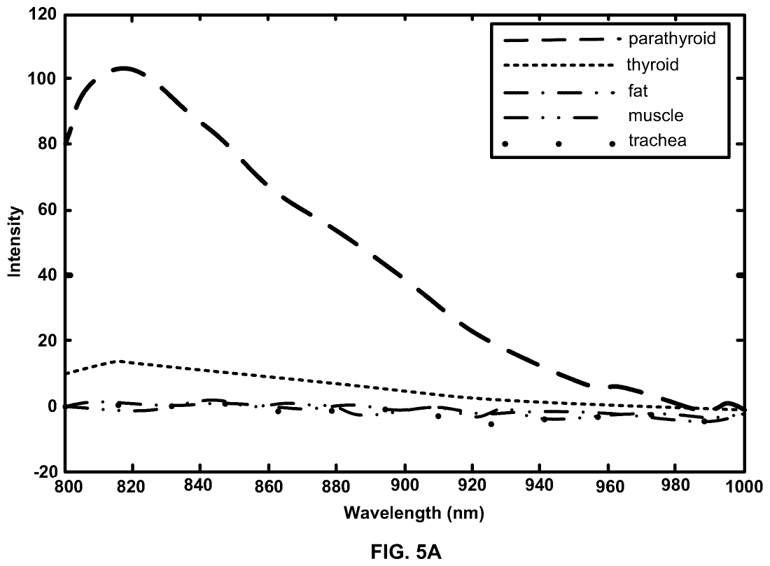

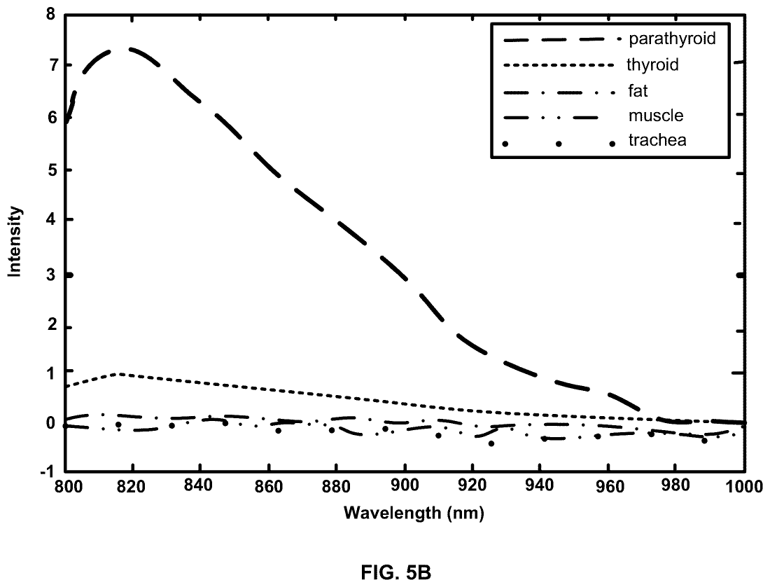

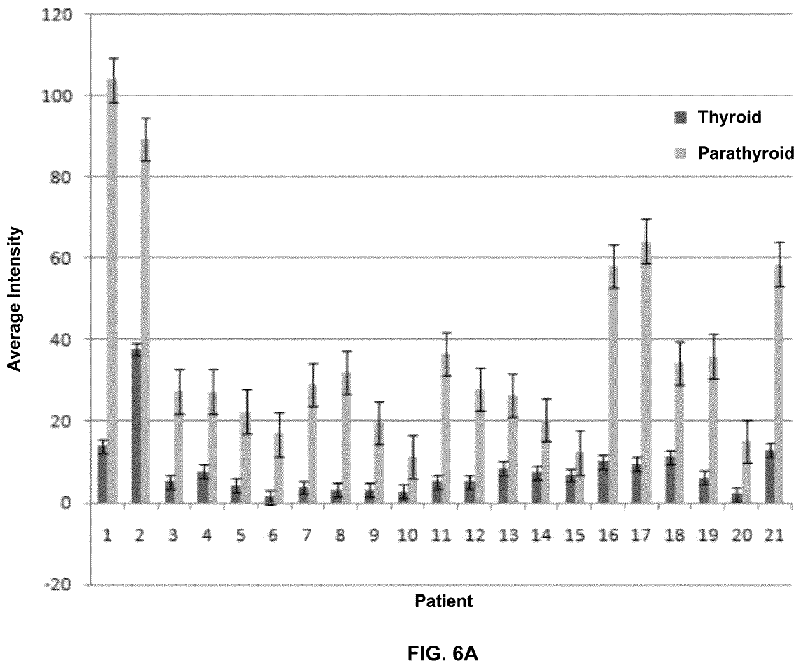

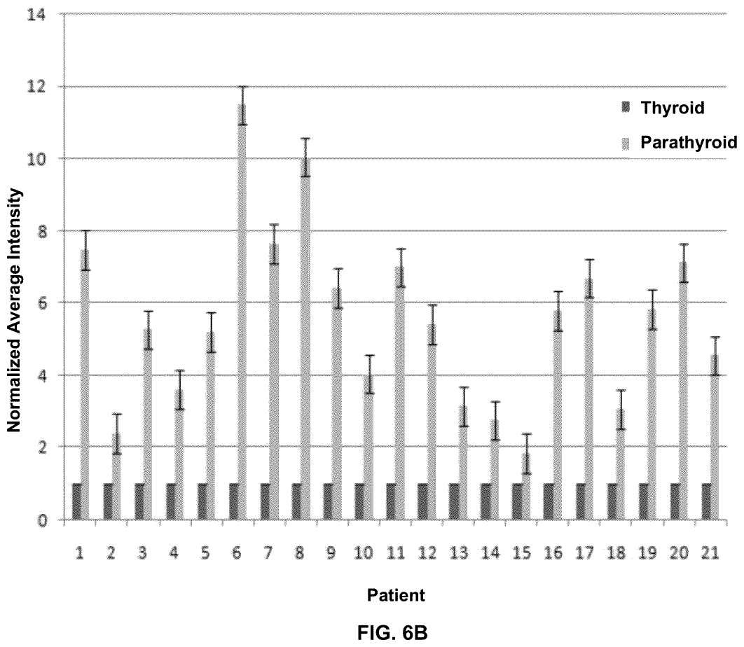

Auto-Fluorescence Measurements. Now also referring to FIGS. 5 and 6, clinical measurements were performed. Twenty-one patients ages 18-99 regardless of race and gender were included in the study under informed written consent. All patients with primary thyroid or parathyroid pathophysiology undergoing thryoidectomy or parathyroidectomy were considered. An initial evaluation was conducted by the participating endocrine surgeon while seeing the patients at the Vanderbilt Clinic. Final eligibility was determined in the preoperative evaluation based on clinical condition and safety of the patient.

A standardized protocol was followed for all fluorescence measurements from patients in vivo. During each surgery, the sterilized optical fiber probe was placed in contact with various tissues in the exposed neck area, and spectral measurements were acquired from each of those sites. The tissue type was noted, along with the physician's confidence in the investigated sites' histological identity. Spectra were collected using a 300 ms signal integration time. In all cases, the overhead fluorescent lights were turned off during the measurements, and other lights were turned away from the measurement site in case they contained spectral components that could interfere with the results. Any investigated sites that were surgically resected were collected for histological identification and analyzed by a pathologist. In each patient, the auto-fluorescence from the parathyroid was compared to the auto-fluorescence from the thyroid and other tissues in the neck. The signal from the parathyroid gland has the highest peak intensity and is easily distinguishable from the surrounding tissues as seen in FIG. 5. Further, thyroid auto-fluorescence is stronger than surrounding muscle and fat but weaker than the parathyroid. Average peak intensity for parathyroid auto-fluorescence was consistently greater than that of the thyroid and other tissues with a p-value of 0.0001 across all patients shown in FIG. 6. Furthermore, parathyroid is 2-11 times more fluorescent than all other tissues found in the neck across all patients in vivo. Thus, the results from Example 3 are very promising. NIR auto-fluorescence provides an accurate detection of parathyroid tissue intra-operatively.

Statistical Analyses. Clinical spectra were smoothed with an averaging filter of size 10. The 6 measurements from each site were average and then normalized to the mean peak thyroid intensity. A right-tailed student's T-test was used to test for the significance of the increased parathyroid signal over the 21 patients. Values of P≤0.05 were considered to be significant.

Understanding the Results. The results set forth above show that NIR auto-fluorescence spectroscopy according to the invention can successfully detect parathyroid tissue in vivo, in real-time and non-intrusively during endocrine surgery. In each patient, the parathyroid signal is greater than the signal from the thyroid and other tissues in the neck. Moreover, the p-value shows that the differences in signal strength are statistically significant and applicable across all patients and diseases. This finding is very promising for surgical identification. The system discriminates parathyroid glands from the surrounding tissue with high accuracy. Near-infrared auto-fluorescence is quick and relatively cheap to implement compared to other intraoperative localization methods such as a sestamibi scan, which can be very time consuming and expensive. This method improves on the accuracy and sensitivity of visual recognition—a highly subjective measure dependent on the experience of the surgeon. Furthermore, most thyroidectomies and parathyroidectomies are typically performed by surgeons who do not perform a high volume of these surgeries, underscoring the clinical need for an improved method. Thus, the results from Example 3 are very promising. NIR auto-fluorescence provides an accurate detection of parathyroid tissue intra-operatively.

### Embodiment 4

This example in part illustrates an apparatus or system, which can be utilized to practice a method or process according to one embodiment of the invention.

Referring now to FIG. 3G, a system 300G is schematically shown. The system 300G has a light source 352, which is designed to provide a beam of light with appropriate excitation wavelengths and energy so as to excite certain molecules of sample tissue 307, when receiving the beam of light, to generate fluorescence emission. In this embodiment, the light source 352 is a diode laser capable of generating a beam of laser light with wavelength 785 nm and power 80 mW. Other suitable light sources can also be utilized to practice the invention.

A first optical fiber 354 optically couples the light source 352 with a probe 356 that functions as a light delivery and collection device. In this embodiment, the first optical fiber 354 is a 300 μm fiber. The probe 356 receives a beam of light 303 from the light source 352 through the first optical fiber 354 and delivers it to the tissue 307 through head portion 358. The head portion 358 also collects optical signals 305 emitted from the tissue 307 responsive to the incident beam of light 303. In one embodiment, the head portion 358 is configured with a noise filter 358a and a band pass 358b to maintain the quality of the optical signals.

A second optical fiber 360 optically couples the probe 356 to means for recording, processing and displaying the optical signals, which, in one embodiment, includes a spectrograph 366, a CCD 368 and a computer 370 with display, where the CCD 368 is in communication with both spectrograph 366 and computer 370. The spectrograph 366 may have internal noise filter installed therein. The second optical fiber 360 is optically coupled with the spectrograph 366 through a coupler 364. Each of the first optical fiber 354 and the second optical fiber 360 may comprise one or more fibers. In one embodiment, the second optical fiber 360 has 6 or 7 of 300 μm fibers arranged in an array.

CCD 368 and computer 370, adapted with appropriate controller cards and software to control signal acquisition, process data and display spectra, are utilized to process and display images corresponding to the optical signals 3 05 emitted from the tissue 307, which may guide a surgeon or a medical professional through a medical procedure to identify tissues and act on them accordingly.

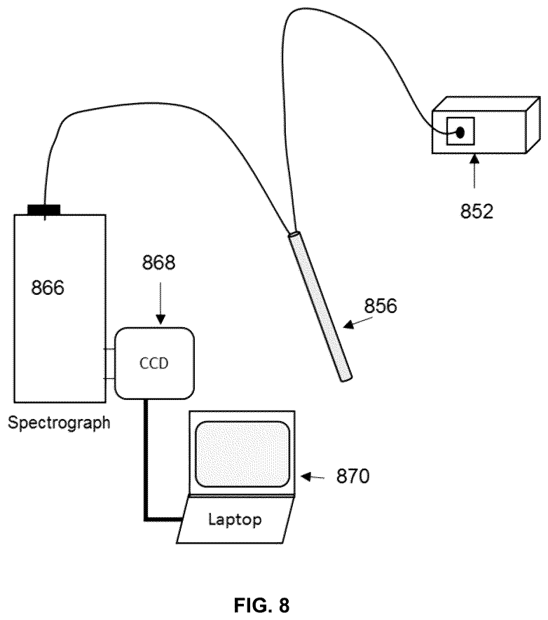

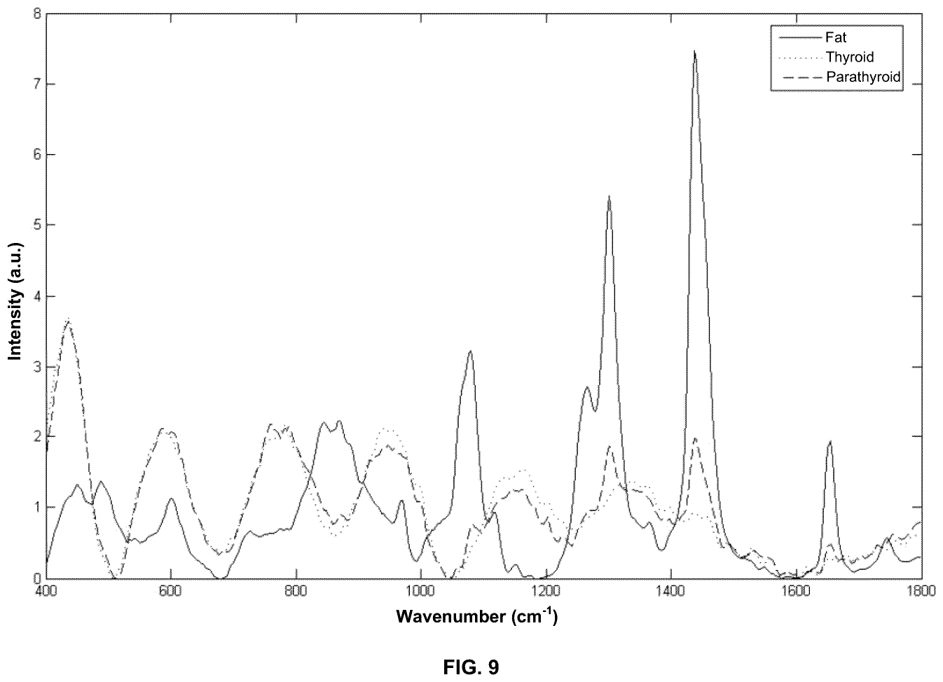

Now referring to FIGS. 8 and 9, FIG. 8 is a photo of a clinical Raman system reduced to practice, which is corresponding to system 300G of FIG. 3G. As previously discussed, Raman scattering is a very weak phenomena, and therefore practical measurement of Raman spectra of a medium requires high power excitation laser sources and extremely sensitive detection hardware. After detection, post processing techniques are required to subtract the fluorescence background and enable accurate visualization of the Raman spectra. FIG. 9 shows processed Raman spectra of various tissue types. As shown, Raman spectra are plotted as a function of frequency shift in units of wavenumber (cm⁻¹). The region of the Raman spectra where most biological molecules have Raman peaks is from 500 to 2000 cm⁻¹. In contrast to fluorescence spectra, Raman spectra have sharp spectral features that enable easier identification of the constituent sources of spectral peaks in a complex sample. In the context of detecting the changes that cancerous tissues undergo, differences in the Raman spectral features that correlate to the increased nucleic acid content in neoplastic cells has been observed.

Patients were recruited and an initial evaluation was conducted while seeing the patients at the Vanderbilt Clinic. Each patient's final eligibility for participating in the study was determined in the preoperative evaluation deeming the individual as a safe and acceptable candidate. Only adult patients between the ages of 18-99 years with primary thyroid or parathyroid pathophysiology undergoing thryoidectomy or parathyroidectomy were considered.

During the operations, the sterilized optical probe was placed on various tissues in the exposed neck area and spectral measurements were acquired from each of those sites. The tissue type was noted, as well as the physician's confidence in the investigated sites' histological identity. Spectra were collected using a 3 second signal collection time. If the signal saturated the fiber optic probe was kept in contact with the tissue and the signal collection time was reduced to 1 second. If saturation was observed at 1 second the signal collection time was again reduced to 0.1 second. In all cases, the overhead fluorescent lights were turned off during the measurements. Any luminescent lights left on were turned away from the measurement site. When medically appropriate, specimens from which measurements are taken were then collected for histological identification. All specimens collected were processed and analyzed by a pathologist.

For most samples, the raw spectra collected in vivo exhibited the distinct signal saturation in measurements of the parathyroid glands. The raw spectra from parathyroid samples saturated times out of 40 measurements, or 87.5%. The signal saturated from the thyroid in only 17 of 49 measurements, or 34.7%. The signal from nerve, fat, and lymph node was not observed to have saturated. While the signal intensity varies slightly from measurement to measurement, on average the raw signal from the parathyroid is at least two times greater than that of the thyroid and the other surrounding tissues measured. This difference in intensity of the signals was seen immediately after the measurement was taken intraoperatively.

This study aimed to investigate if parathyroid tissue has unique optical properties that could be used to identify the parathyroid glands and differentiate them from neck background intraoperatively. We assessed the capabilities of Raman spectroscopy to accomplish this differentiation. Preliminary studies clearly indicated the potential for identifying the various tissues of the neck. Measurements taken of the tissue in the neck, including parathyroid, thyroid, lymph nodes, nerve, and fat, indicated these tissues have unique optical properties. The raw signal from parathyroid saturates while this was not observed as often in other tissues.

### Example 2

### Intraoperative Assessment of Parathyroid Gland Vascularity Using Laser Speckle Contrast Imaging

The LSCI technique analyzes the interference pattern produced when coherent light is incident on a surface. Minute differences in path length created by the light waves scattering from different regions of the surface produce bright and dark spots of constructive and destructive interference respectively, termed as a speckle pattern. This speckle pattern fluctuates depending on how fast particles are moving within a few microns of the surface. Blurring of the speckle pattern occurs when the motion is fast relative to the integration time of the detector. Analyzing this spatial blurring provides contrast between regions of faster versus slower motion and forms the basis of LSCI. This technique is sensitive to microvascular perfusion and has been employed in a variety of tissues where the vessels of interest are generally superficial, such as the retina, skin and brain. Many of its applications have been in laboratory settings for investigating phenomena such as skin blood flow dynamics in response to external stimuli, and cerebral blood flow in animal models of stroke. Clinically, LSCI and similar techniques have been applied to monitoring Port Wine Stain laser therapy, correlating perfusion with healing time in burn wounds, and measuring cerebral blood flow in patients undergoing brain tumor resection. However, there are no published reports on the application of LSCI or any other label-free optical method for assessing parathyroid gland viability during endocrine surgery. Parathyroid glands are densely packed with blood vessels, given that they secrete PTH to the entire body. Furthermore, their small size (3-8 mm) makes many of these vessels superficial, making these glands suitable targets for assessment using LSCI.

Embodiments of this invention present the LSCI for real-time assessment of parathyroid viability. Taking an experienced surgeon's visual assessment as the gold standard, LSCI can be used to distinguish between well vascularized (n=32) and compromised (n=27) parathyroid glands during thyroid surgery with an accuracy of 91.5%. Ability to detect vascular compromise with LSCI was validated in parathyroidectomies. Results showed that this technique is able to detect parathyroid gland devascularization before it is visually apparent to the surgeon. Measurements can be performed in real-time and without the need to turn off operating room lights. LSCI shows promise as a real-time, contrast-free, objective method for helping reduce hypoparathyroidism after thyroid surgery.

One aspect of the invention relates to a method of using LSCI for intraoperative assessment of parathyroid gland viability in a surgery. The method includes diffusing a beam of light onto a tissue surface of a parathyroid gland of a patient to illuminate the tissue surface; acquiring images of the illuminated tissue surface, where each of the acquired images comprises a speckle pattern; and processing the acquired images to obtain speckle contrast images for the intraoperative assessment of parathyroid gland viability. In the speckle contrast images, a perfused parathyroid gland has low speckle contrast, and a devascularized parathyroid gland has high speckle contrast.

In one embodiment, the method further includes displaying the speckle contrast images of the parathyroid gland in real-time.

In one embodiment, the beam of light is emitted from a light source. In one embodiment, the light source comprises an infrared laser.

In one embodiment, said acquiring the images of the illuminated tissue surface is performed with a detector.

In one embodiment, the detector comprises a camera. In one embodiment, the camera comprises an infrared camera or a near-infrared camera. In one embodiment, the detector further comprises a focus tunable lens attached to the front end of the camera.

In one embodiment, said processing the acquired images comprises calculating a plurality of speckle contrasts from the acquired images of the parathyroid gland.

In one embodiment, said calculating plurality of speckle contrasts comprises defining a window with a number of pixels over which a speckle contrast is to be calculated; moving the window across the acquired image of the speckle pattern; and at each location, calculating the speckle contrast as a standard deviation of pixel intensity values σₛ within the window divided by a mean intensity value [symbol] I [symbol] as follows:

$${K}_{s} = \frac{{σ}_{s}}{〈 I 〉}$$

where the resultant speckle contrast image has values that range from 0 to 1, with values closer to 0 representing regions of greater motion (perfusion) and 1 representing regions with no motion.

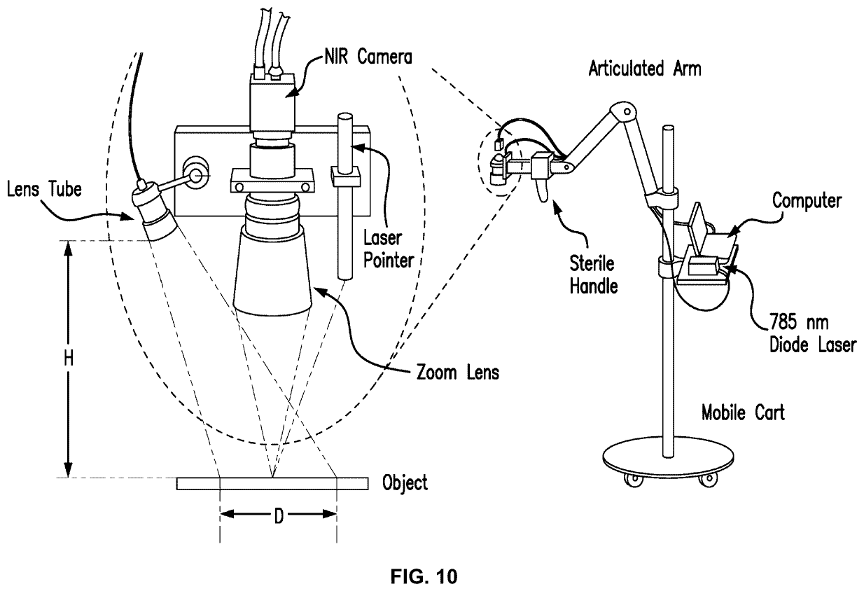

Another aspect of the invention relates to an apparatus (device/system) of using LSCI for intraoperative assessment of parathyroid gland viability in a surgery. In one embodiment, as shown in FIG. 10, the apparatus includes a light source (e.g., a 785 nm diode laser) for emitting a beam of light, and a diffuser (e.g., a lens tube having a biconvex lens) coupled to the light source for diffusing the beam of light onto a tissue surface of a parathyroid gland (e.g., object) of a patient to illuminate the tissue surface. The biconvex lens diffuses the beam of light to a spot with a diameter of D on the tissue surface at a distance of H from the edge of the biconvex lens. The light source is an infrared laser, or the like.

The apparatus also includes a detector (e.g., NIR camera) positioned above the tissue surface for acquiring images of the illuminated tissue surface. In one embodiment, the detector comprises a camera. The camera can be a charge-coupled device (CCD) camera and/or a complementary metal-oxide-semiconductor (CMOS) camera. In one embodiment, the camera comprises an infrared camera or a near-infrared (NIR) camera. In one embodiment, the detector further comprises a focus tunable lens (e.g., a zoom lens) attached to the front end of the camera.

The apparatus further includes a computer (controller) arranged to operate the detector to acquire the images of the illuminated tissue surface, receive the acquired images from the detector, and process the acquired images to obtain speckle contrast images for the intraoperative assessment of parathyroid gland viability. In the speckle contrast images, a perfused parathyroid gland has low speckle contrast, and a devascularized parathyroid gland has high speckle contrast.

The apparatus also includes a display for displaying the speckle contrast images of the parathyroid gland in real-time.

In addition, the apparatus includes a laser pointer arranged in relation to the detector such that its beam is co-localized with a center of the field of view of the detector at a distance.

In one embodiment, said processing the acquired images of the parathyroid gland by the controller is performed with calculating a plurality of speckle contrasts from the acquired images of the parathyroid gland.

In one embodiment, said calculating a plurality of speckle contrasts from the acquired images of the parathyroid gland is performed by defining a window with a number of pixels over which a speckle contrast is to be calculated; moving the window across the acquired image of the speckle pattern; and at each location, calculating the speckle contrast as a standard deviation of pixel intensity values σₛ within the window divided by a mean intensity value [symbol] I [symbol] as follows:

$${K}_{s} = \frac{{σ}_{s}}{〈 I 〉}$$

where the resultant speckle contrast image has values that range from 0 to 1, with values closer to 0 representing regions of greater motion (perfusion) and 1 representing regions with no motion.

In certain aspects, this invention uses laser speckle imaging to provide a real-time non-invasive means to inform the surgeons whether or not a parathyroid gland is still being perfused and is therefore viable. During a thyroid or parathyroid surgery, the invented apparatus or device acquires and processes images of the parathyroid gland. The device comprises a 785 nm wavelength laser and a near-infrared camera with a zoom lens, positioned above the surgical filed via an articulated arm. The laser light is diffused onto the tissue surface and images are acquired by the camera. Images are acquired using a program developed on commercially available software, and processed to produce speckle contrast image through the lab-developed dynamic link library file written in C++. This allows the speckle contrast images to be displayed to the surgeon in real-time (as they are acquired). In these speckle contrast images, perfused (and therefore viable) parathyroid glandss generally have low speckle contrast, while devascularized parathyroid glands have high speckle contrast.

The device and processing software have been proven feasible in a group of patients. Speckle contrast images of vascularized parathyroid glands and devascularized parathyroid glands are acquired during parathyroidectomy, where darker regions represent areas of greater blood flow and lighter regions represent areas of reduced blood flow. The speckle contrast images of parathyroid glands are compared against the surgeon's assessment of whether or not the gland was devascularized, where the confidence level of the surgeon were also recorded. For larger parathyroids the images agree 100% with the surgeon's assessment, however the device did not function as well for smaller (normal) glands. In certain embodiments, some modifications to the device is made to incorporated a zoom lens, which is now getting better agreement for smaller parathyroid glands.

Conventional laser speckle contrast imager appears less ergonomically suitable (more bulky) for use in the OR in the assessment of parathyroid glands than the invented LSCI device. According to the invention, the LSCI device can be combined with another lab-built device for detecting parathyroid gland using fluorescence, and marketed to endocrine surgeons. In addition, the invention allows for a more compact final product capable of assisting surgeons in first identifying parathyroid glands, and then assessing their viability.

This exemplary example was performed in patients undergoing thyroidectomies and parathyroidectomies by measuring the speckle contrast of parathyroid glands in vivo. Performance of LSCI was evaluated in thyroidectomies, by grouping speckle contrast from normal functioning and compromised parathyroid glands according to the surgeon's classification. Validation was performed in parathyroidectomies, where the state of vascularity of the parathyroid gland was controlled in glands planned for excision. Results demonstrate the effectiveness of LSCI for parathyroid gland assessment.

### Materials and Methods

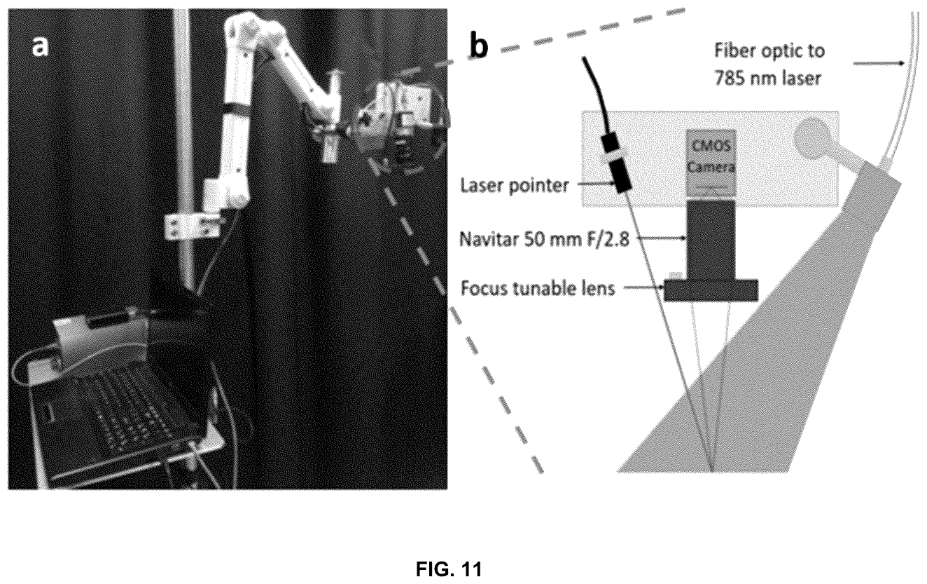

LSCI System Design: An LSCI system, as shown in FIG. 11, which is operably the operating room (OR), was developed according to one embodiment of the invention. In the exemplary embodiment, the LSCI system is mounted on an articulated arm attached to a mobile cart. A 785 nm diode laser (Innovative Photonics Solutions, Monmouth Junction, NJ) is coupled through a 400 μm fiber optic patch cord (Thorlabs, Newton, NJ) to a lens tube containing a 75 mm focal length biconvex lens (Thorlabs, Newton, NJ). This lens diverges (diffuses) the laser light to a spot of about 8 cm diameter at a distance of about 45 cm from the edge of the lens. The irradiance at the surface was measured to be about 0.6 mW/cm². A near-infrared optimized camera (acA1300-60gmNIR, Basler AG, Ahrensburg, Germany) captures the images, which are focused onto the camera sensor by an imaging lens system (Navitar 50 mm F/2.8, Navitar, Woburn, MA). Attached to the front end of the imaging lens system is a focus tunable lens (EL-16-40-TC-VIS-5D-M27, Optotune, Dietikon, Switzerland). This allows the images to be focused during each procedure from outside the sterile field as the height of the operating table may be slightly adjusted during surgery. The field of view of the camera was measured to be about 5 cm×6 cm. A 5 mW 660 nm laser pointer (DigiKey, Thief River Falls, MN) is attached such that its beam is co-localized with the center of the camera's field of view at a distance of 45 cm. The purpose of this laser pointer is to guide the surgeon in positioning the system above a parathyroid gland so that it is approximately in the center of the field of view. Images recorded by the camera are sent to a laptop computer for processing and display.

Ensuring Adequate Sampling of Speckle Pattern: An important consideration when performing LSCI is that the speckle pattern must be adequately sampled. The smallest speckle should be at least twice the size of the sensor pixel in order to meet the Nyquist sampling criterion and avoid underestimating speckle contrast. To test whether the invented LSCI system met this criterion, an optical tissue phantom made of polydimethylsiloxane (PDMS) with titanium dioxide to simulate tissue scattering (reduced scattering coefficient of 8 cm⁻¹) was illuminated and imaged with the LSCI device using different aperture sizes. The power spectrum was analyzed to ensure there was no aliasing. The iris size was set close to f/16 and locked in this position for the entire study. A well-defined energy band centered at the origin of the power spectrum confirmed no aliasing. Although larger apertures also had no aliasing, this aperture size was chosen because it also resulted in very little background from ambient lighting.

Patient Recruitment and Imaging Protocol: This study was conducted in accordance with the Declaration of Helsinki and its amendments. The study was approved by the Vanderbilt University Medical Center (VUMC) Institutional Review Board (IRB). Patients undergoing partial or total thyroidectomy at the Vanderbilt University Medical Center were recruited and written informed consent was obtained from each patient (n=20) prior to participation.

Images of parathyroid glands were recorded during the course of surgery at the discretion of the surgeon. The surgeon first determined the state of vascularity of the gland based on visual inspection, and then positioned the LSCI device above the surgical field so that the laser pointer beam was on the parathyroid gland. With the camera integration time set to about 50 ms to allow imaging with the room lights on, white light images were initially acquired to identify the location of the parathyroid gland. The laser pointer was turned off and the integration time was then set to 5 ms for the rest of the image acquisitions. This integration time is within the range typically used for LSCI, and results in the room lights contributing very little signal to the image while effectively detecting the 785 nm speckle pattern. Next, the 785 nm laser was turned on and images were acquired and sent to the computer for real-time processing of speckle contrast images. Roughly fifteen seconds of acquisition were allowed for each gland to ensure that a sufficient number of images was obtained. The surgeon was blinded to all images during the surgery.

Validation of Technique: Given that there is no gold standard for assessing parathyroid gland viability, validation of this technique was performed as follows. Patients undergoing parathyroidectomy at the VUMC were recruited and written informed consent obtained prior to participation following IRB approval. In parathyroidectomies, a diseased parathyroid gland is planned for removal. As part of standard procedure, the surgeon ligated blood supply to the diseased parathyroid gland prior to excision. This provides a controllable scenario for evaluating the capability of the device to detect parathyroid gland vascular compromise.

Speckle contrast images were acquired before and less than one minute after the surgeon ligated the blood supply to the gland, in preparation for removal. To confirm removal of the hyperactive parathyroid gland, intraoperative measurement of PTH was performed before and 10 minutes after excision of the gland. Changes in speckle contrast between the vascularized and ligated state were assessed.

Speckle Contrast Calculation: The number of pixels over which spatial speckle contrast is calculated is important, with too few pixels resulting in inaccuracy in contrast estimation while too many pixels sacrificing spatial resolution. Calculating speckle contrast over a 5×5 or 7×7 pixel region is generally considered a good compromise. In this exemplary embodiment, a 5×5 pixel window was used in calculating speckle contrast. The window is moved across the image of the acquired speckle pattern and at each location, the speckle contrast is calculated as the standard deviation of pixel intensity values within the window divided by the mean (Equation 1). The resultant speckle contrast image has values that range from 0 to 1, with values closer to 0 representing regions of greater motion (perfusion) and 1 representing regions with no motion. Image acquisition and display is performed through a custom LabVIEW program, while a custom dynamic link library file enables processing and display at about 10 frames per second.

$${K}_{s} = \frac{{σ}_{s}}{〈 I 〉} \qquad ( 1 )$$

Data Analysis: Data was analyzed using MATLAB R2015a (The MathWorks Inc., Natick, MA). For each set of recordings on a single parathyroid gland, 20 speckle contrast images were averaged after acquisition to improve the spatial resolution of the final image. To correct for slight movement of the surgical field during recordings, an image registration algorithm was employed before averaging. Any image with sudden jerky motion (visually identified as a global sharp decrease in speckle contrast) was excluded from the averaging. The parathyroid gland was then demarcated using the “roipoly” function, avoiding bright spots caused by specular reflection, and the average speckle contrast within this region was calculated. In certain embodiments of the device, polarizers are used to reduce specular reflection. The data was then grouped into vascularized and compromised according to the surgeon's assessment of the gland. Compromised parathyroid glands were also further grouped into those that the surgeon decided to transplant and those that were left in place. Tests for statistical significance were performed using a two-sided two sample Student's t-test and p-values less than 0.01 were considered statistically significant.

Effect of Ambient Lighting on Speckle Contrast Images: Performing measurements without the need to turn off the OR lights would minimize disruption to the surgical workflow. A microfluidic flow phantom made of PDMS and titanium dioxide to achieve a reduced scattering coefficient of 8 cm⁻¹ was used to evaluate the effect of ambient lighting on speckle contrast images in the laboratory. The device had a 400 μm channel through which a 1% Intralipid (Sigma-Aldrich, St. Louis, MO) solution was flowed. Images were acquired with the room lights on and then off, and 20 speckle contrast images were averaged for each condition.

For one patient undergoing parathyroidectomy, two sets of measurements on one parathyroid gland were obtained to determine the effect of the OR lights on speckle contrast images. The surgeon first positioned the device so the parathyroid gland was in the field of view. Images were recorded with the operating room lights on, but with the surgical lights above the operating table pointing away from the surgical field. Then, without moving the device, all operating room lights were turned off and another set of images were obtained. For each condition, 20 speckle contrast images were averaged and the same region of interest was used to calculate average parathyroid gland speckle contrast.

### Results

Device Design: A portable, clinical LSCI device/system was developed and is shown in FIG. 11. In essence, the device includes a 785 nm coherent light source to diffuse light onto the surgical field, a near-infrared optimized camera to record the speckle pattern produced, and a computer to process and display images at 10 frames per second. The field of view was measured to be about 5 cm×6 cm at a distance of 45 cm above the operating table and the laser intensity at this distance was 0.6 mW/cm².

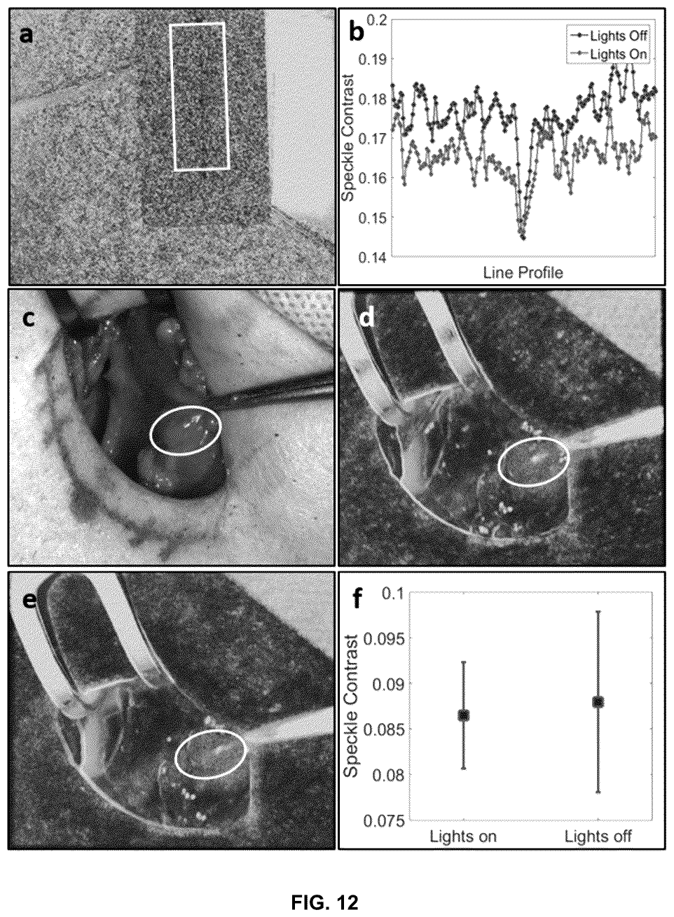

Effect of Ambient Lighting on Speckle Contrast Images: Performing measurements without the need to turn off the operating room (OR) lights is essential so that any disruption to the surgical workflow is minimized. The effect of ambient lights on speckle contrast was evaluated in the laboratory on a microfluidic flow phantom with a 1% Intralipid solution flowing through its 400 μm channel (panels a-b of FIG. 12). A rectangular region of interest crossing the channel was selected and line profiles perpendicular to the channel within this region were averaged for both conditions, lights on and lights off. There was no significant difference in the decrease in speckle contrast when crossing the channel, using a two-sample Student's t-test (p=0.19).

To determine how room lights would affect LSCI data acquired in the operating room, the system was tested in vivo on a patient undergoing parathyroidectomy. One set of images was acquired with the room lights on and the surgical lights above the operating table pointed away from the surgical field, and another set with all lights off. For each condition, 20 speckle contrast images were averaged after acquisition and the same region of interest was used to calculate average parathyroid gland speckle contrast. There was a 1.7% decrease in the average speckle contrast value of the parathyroid gland when the OR lights were left on, compared to when they were off (panels c-f of FIG. 12). This is to be expected since OR lights have a broad frequency spectrum and therefore do not produce a speckle pattern. The uniform lighting fills in dark spots in the speckle pattern generated by the laser, thereby reducing the contrast. However, the magnitude of this decrease (about 0.002) was miniscule compared to the possible range of speckle contrast values (0 to 1). Additionally, this value is less than 2% of the range of average parathyroid gland speckle contrast obtained in this study. These results indicated that the performance of the LSCI system is not affected by ambient light and validates its application in the operating room with the OR lights on but pointed away from the patient during image acquisition.

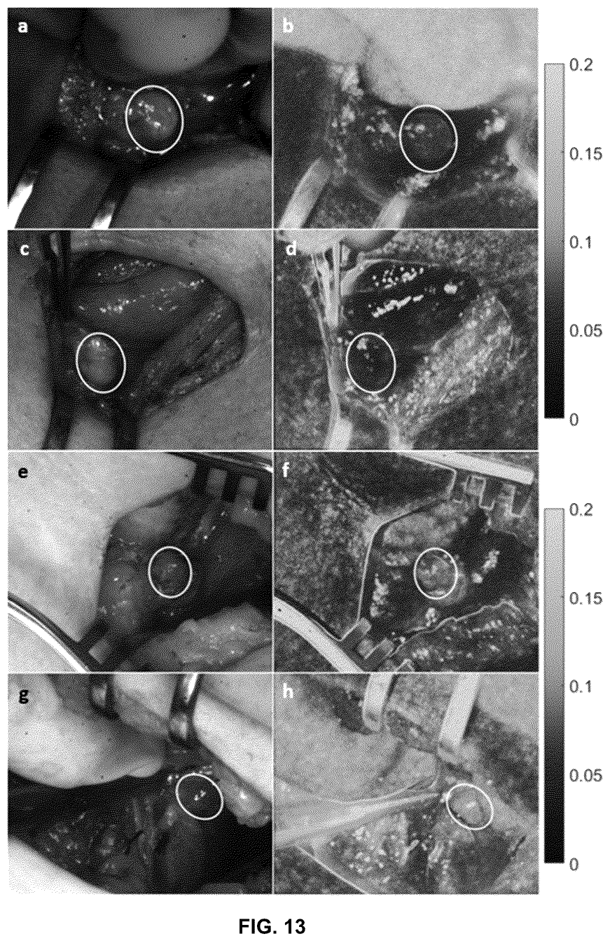

Vascularized vs. Compromised Parathyroid Glands: The LSCI system was tested in twenty patients undergoing thyroidectomy at Vanderbilt University Medical Center. Speckle contrast images of parathyroid glands were acquired during the course of surgery and at the discretion of the participating surgeon. The surgeon's visual assessment of the gland's viability (without input from speckle contrast images) was recorded at the time of image acquisition for each gland. FIG. 13 shows examples of parathyroid glands considered vascularized and compromised. The left column shows white light images of the surgical field, and the right column shows the corresponding speckle contrast image (average of 20 images). Parathyroid glands are indicated with a white ellipse. As these images show, parathyroid glands considered vascularized by the surgeon have lower speckle contrast than those considered to be compromised.

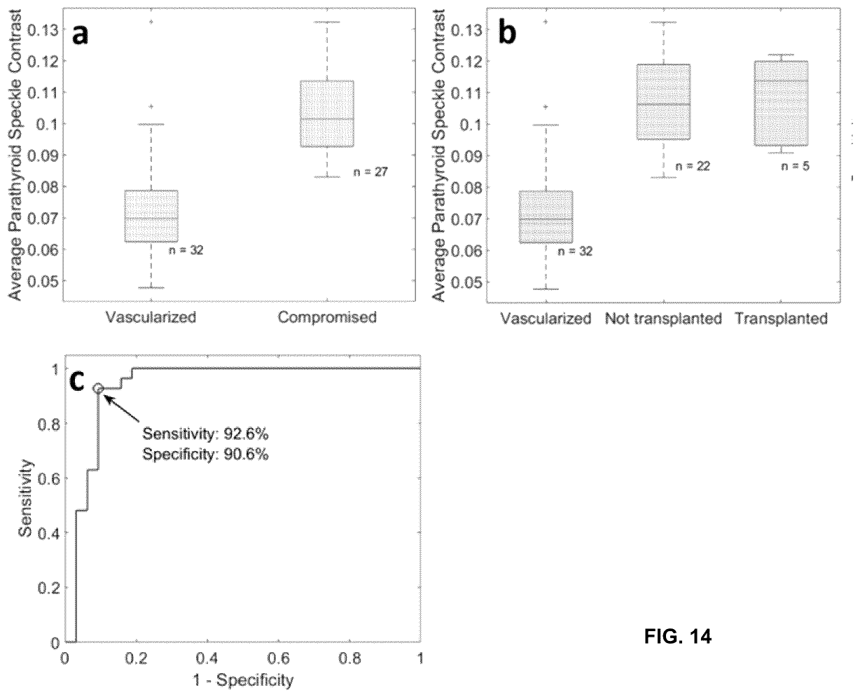

The average speckle contrast within each parathyroid gland was calculated, avoiding bright spots of specular reflection. This data was then grouped according to the surgeon's visual assessment of vascularity, which served as the gold standard. Using a two-sample two-sided Student's t-test, a statistically significant difference (p<0.0001) was observed in the speckle contrast between glands considered vascularized versus compromised as determined by the surgeon (panel a of FIG. 14). Within the compromised group, there were 5 glands that were transplanted, and 22 others that the surgeon decided could be left in place without significant adverse effects to the patient. While the mean speckle contrast was higher in the transplanted group, this difference was not found to be significant using a two-sample t-test. These results are shown in panel b of FIG. 14.

Using speckle contrast as a classifier to distinguish between the vascularized and compromised parathyroid glands, a receiver operating characteristic (ROC) curve was generated with an area under the curve of 0.935 (panel c of FIG. 14). The optimum threshold for distinguishing between the two groups was found to be 0.09, which resulted in a sensitivity of 92.6% (25/27 compromised glands correctly identified) and a specificity of 90.6% (29/32 vascularized glands correctly identified). From this, the overall accuracy was calculated to be 91.5%.

Validation of Technique: Given that there is no gold standard for assessing parathyroid gland viability and these normal functioning glands cannot be excised for histological validation without compromising patient care, validation of the LSCI system for this application was performed on eight patients undergoing parathyroidectomy, where a diseased parathyroid gland is planned for removal. As part of standard procedure, the surgeon ties off all blood supply to the gland prior to excision. This provides a controllable scenario for evaluating the capability of the device to detect when a parathyroid gland is compromised as well as to determine the detection limit (or time) of the system. To confirm removal of the hyperactive parathyroid gland, intraoperative measurement of PTH is routinely performed before and 10 minutes after excision of the gland.

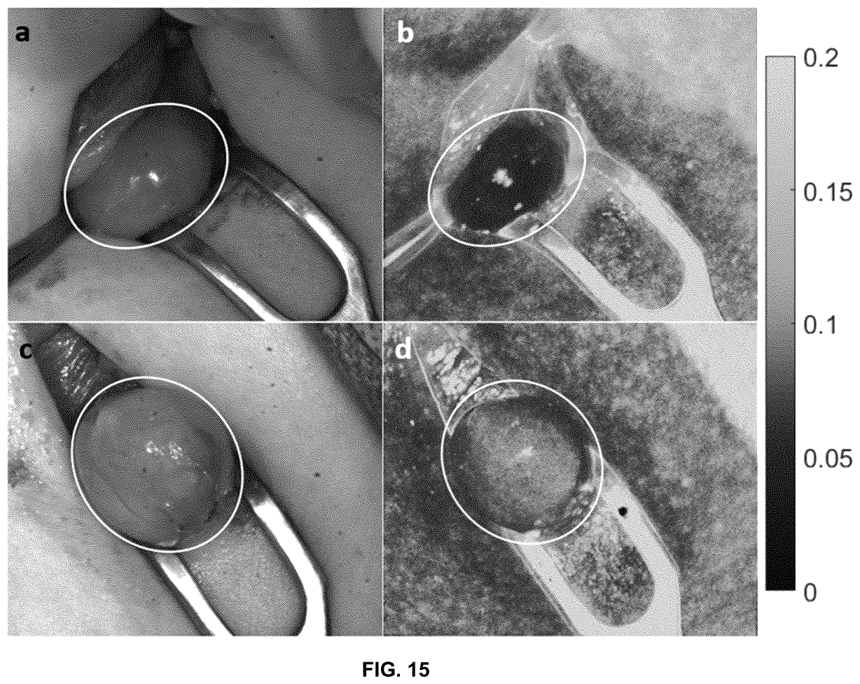

An example of a hypercellular diseased parathyroid gland marked for removal is shown in FIG. 15. Again, the left column shows white light images while the corresponding speckle contrast images are in the right column. Images on the top row are of the gland before the surgeon tied off the blood supply. Images on the bottom row were taken on average 30 seconds after the surgeon tied off the blood supply to the gland. According to the surgeon and as seen in the white light image, this gland did not appear visually different from its initial state. However, there is a clear change in the speckle contrast images. In all cases, speckle contrast increased by a minimum of 18% after the surgeon tied off the blood supply (data shown in Table I; mean increase of 0.043). Further, post-excision intraoperative PTH was also lower than pre-excision PTH, confirming removal of the diseased gland. PTH data was not acquired for two glands of patient 5 because removal of the first diseased gland resulted in PTH falling within the acceptable range (10-70 pg/mL) and all glands were diseased (Roche Elecsys 2010).

TABLE I
Summary of data from excised diseased parathyroid glands.
In all cases there was a large increase in speckle contrast
seconds after ligation of the blood supply to the gland. There
was also a decrease in PTH related to the number of diseased
glands removed. In patient 5, LSCI data was obtained from
3 glands that were excised, however PTH measurement was
not performed after removal of glands 2 and 3.

| | Pre-ligation | Post-ligation | Pre-excision | Post-excision |
|---|---|---|---|---|
| Patient | speckle | speckle | [PTH] | [PTH] |
| No. | contrast | contrast | (pg/mL) | (pg/mL) |
| 1 | 0.076 | 0.126 | 86 | 26 |
| 2 | 0.057 | 0.094 | 142 | 34 |
| 3 | 0.062 | 0.131 | 172 | 32 |
| 4 | 0.074 | 0.130 | 119 | 115 |
| 4 | 0.069 | 0.119 | 115 | 45 |
| 5 | 0.064 | 0.093 | 151 | 53 |
| 5 | 0.048 | 0.085 | 53 | N/A |
| 5 | 0.084 | 0.113 | N/A | N/A |
| 6 | 0.063 | 0.092 | 130 | 31 |
| 7 | 0.067 | 0.115 | 76 | 26 |

### Discussions

This exemplary embodiment of the invention demonstrates the capability of LSCI to intraoperatively distinguish between vascularized and compromised parathyroid glands. These measurements can be performed in real-time and with the operating room lights on, minimizing disruption to the surgical workflow. The average speckle contrast value of vascularized parathyroid glands was significantly lower than that of the compromised parathyroid glands, consistent with the understanding that reduced blood flow causes less blurring of the speckle pattern and therefore a higher speckle contrast. Using the ROC curve in panel c of FIG. 14, a speckle contrast value of about 0.09 was found to be optimal in distinguishing between the two groups with 91.5% accuracy. The ability of LSCI to accurately detect compromised vascularity in the parathyroid gland was validated in the parathyroidectomy cases. These images further show that this device is able to detect changes in speckle contrast within seconds of devascularization. This is much earlier than a surgeon would be able to identify based on visual inspection alone which relies on the gland turning dark with deoxygenated blood and losing turgor, a process which can take several minutes and is often missed. Unlike other techniques that have been used to assess parathyroid gland viability, LSCI can be performed non-invasively, with no risk of damage to the parathyroid gland by physical trauma, nor damage to any nearby tissues. It does not require administration of an exogenous contrast agent, which can take 1-2 minutes to achieve optimum circulation. Additionally, multiple measurements can be made on the same gland without risk of toxicity. The device enables real-time assessment by processing and displaying speckle contrast images at 10 frames per second. The images shown in the disclosure were created after acquisition by averaging individual frames in order to improve spatial resolution. However, quantitative information on a region of interest can also be displayed in real-time if desired as this information is generated concurrently with imaging. This technique can be performed with the room lights on, only requiring that the lights above the operating table be pointed away from the surgical field.

The biggest challenge facing this study is the fact that the current gold standard for assessing parathyroid gland viability intraoperatively relies on the surgeon's experience which is highly subjective. The participated surgeon has multiple years of experience in this field and is therefore more likely than not to provide accurate assessments. However, thyroidectomies are routinely performed by general surgeons and residents, and a higher error rate is likely, indicating the need for objective assessment of parathyroid viability. These results demonstrate that LSCI has the potential to become the gold standard in this field.

In the absence of an objective gold standard to validate the performance of this device, paired measurements were made in parathyroidectomy cases where the state of vascularity was controlled. It should be noted that the PTH measurements made after excision of the glands are not meant to directly correlate with speckle contrast values but are simply to confirm that the excised tissues are diseased parathyroid glands. A more robust validation method would be to measure PTH after tying off the gland but before excision. However, this was not feasible as it takes about 5-10 minutes for PTH levels to stabilize to a new value and such a procedure would extend OR time and lead to added risk for the patient.

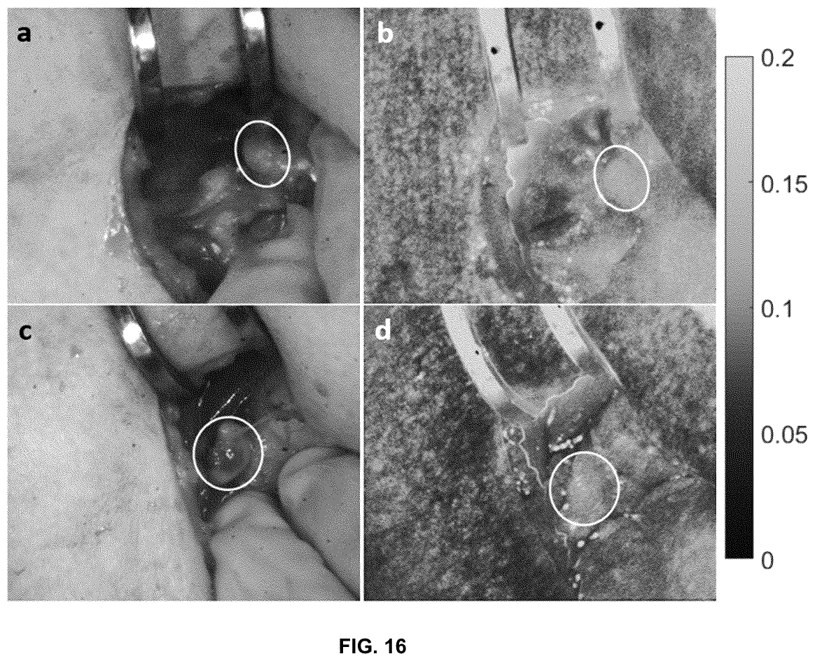

While this device performed with high accuracy in distinguishing between vascularized and compromised parathyroid glands in thyroidectomies, there were 5 instances of disagreement with the surgeon. One case shed light on a possible reason for this disagreement. This particular parathyroid gland was evaluated by the surgeon to be initially well vascularized, however its high speckle contrast value suggested otherwise as observed in panel b of FIG. 16. After 15 minutes, the surgeon re-evaluated this gland as devascularized and transplanted it, with speckle contrast images agreeing with this assessment (pane; d of FIG. 16). This case further demonstrates the ability of LSCI to detect vascular compromise before it is visually apparent to the surgeon.

An interesting observation was that of the 27 glands the surgeon considered to have suffered vascular compromise, only 5 were autotransplanted. The rest were considered capable of recovery. However, no significant differences in speckle contrast were observed between these two groups (though the small sample size for the transplanted group will make any differences difficult to identify). This warrants further investigation with a larger study to identify what is truly viable. Future studies will additionally acquire pre- and post-surgery PTH measurements in patients with autotransplanted and intact parathyroid glands so that patient outcome may be correlated with speckle contrast images. This will enable answering questions such as: “Should all glands with speckle contrast above a given threshold be autotransplanted in order to avoid hypoparathyroidism?”, and/or, “Is it safe to leave suspicious-looking glands in place if at least one parathyroid has speckle contrast below this threshold?”

It should be noted that LSCI senses motion, therefore these measurements are highly susceptible to random movement of the surgical field caused by the patient's breathing and movement of the hands holding retractors to expose the surgical field. Image registration was used to align images prior to averaging, however this does not avoid the artificial decrease in speckle contrast introduced to a single frame by such motion. For reference, the average speckle contrast of an excised parathyroid gland placed on a stable surface in the same operating room was measured to be 0.2, whereas the highest value obtained in vivo was about 0.13. In spite of this unavoidable source of error, very distinct differences in speckle contrast were obtained as demonstrated by the parathyroidectomy data (Table I). To improve the accuracy of the final image, any image with jerky motion (visually identified as a global sharp decrease in speckle contrast) was excluded from the averaging. This was done manually due to the rarity of the event. In the future, should it be required, an algorithm could be employed to automate this process and reject speckle contrast images with values a specified percentage below the mean. Regardless of these limitations, this study clearly shows the applicability of LSCI as a real-time, contrast-free and objective guidance tool for assessing parathyroid gland viability intraoperatively. It will be of particular benefit to less experienced surgeons and residents, and could reduce hospitalization and long-term medication costs associated with post-surgical hypoparathyroidism. This device could also have applicability for other clinical procedures where there is the need to non-invasively investigate the presence of tissue perfusion.

In sum, the invention presents LSCI as a potential real-time, non-invasive, contrast-free and objective tool for guiding surgeons during thyroid surgeries to assess the viability of parathyroid glands. The device according to embodiments of the invention can provide this information to surgeons with minimal disruption to the surgical workflow, and can detect vascular compromise in its early stages before it becomes evident to the surgeon's eye. This tool can therefore significantly minimize post-surgical hypoparathyroidism and its consequent morbidities and costs.

### Example 3

### Intraoperative Assessment of Parathyroid Gland Using Combined Fluorescence and Laser Speckle Contrast Imaging

This example discloses an imaging system capable of performing both auto-fluorescence and laser speckle contrast imaging (LSCI) intraoperatively for guidance in a surgery. This system is developed to guide surgeons performing thyroid and parathyroid surgeries, which allows a surgeon to objectively identify a parathyroid gland during surgery and assess its viability. Auto-fluorescence imaging helps identify the parathyroid, while LSCI helps assess its viability.

To the inventors' knowledge, combination of the two imaging modalities (fluorescence and LSCI) into one clinical instrument according to the invention has not yet been reported at the time when the invention was made.

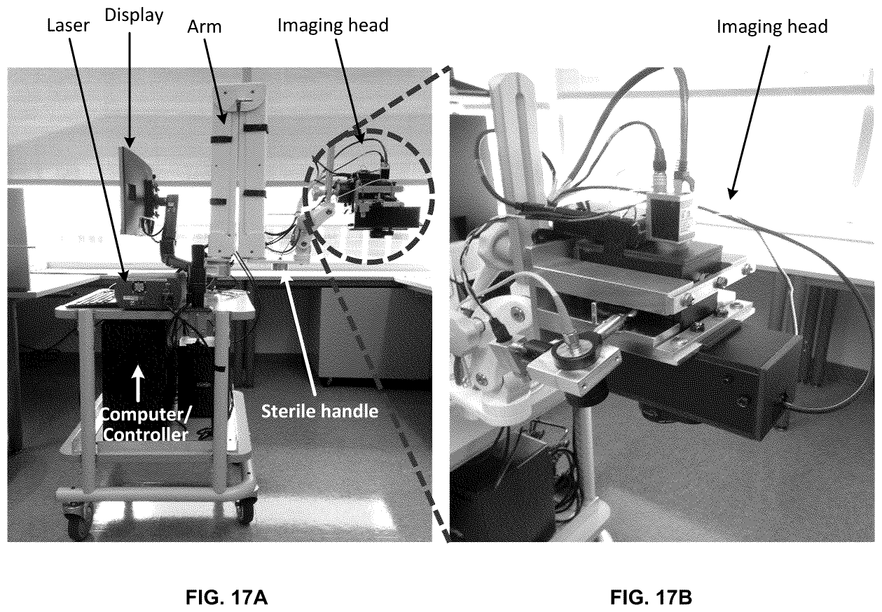

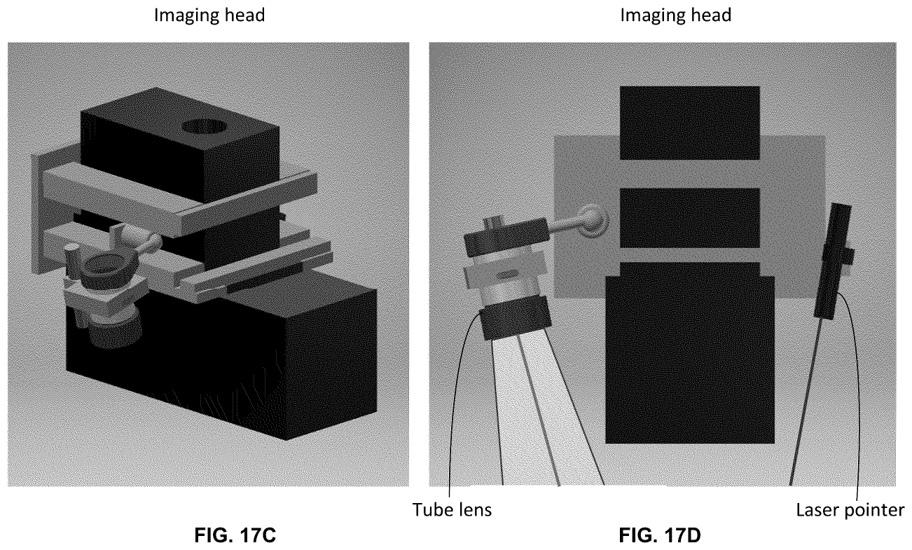

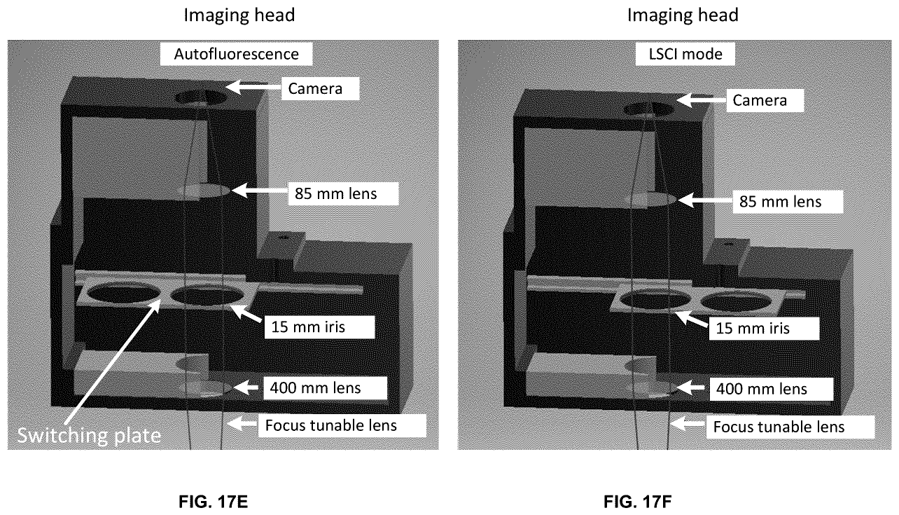

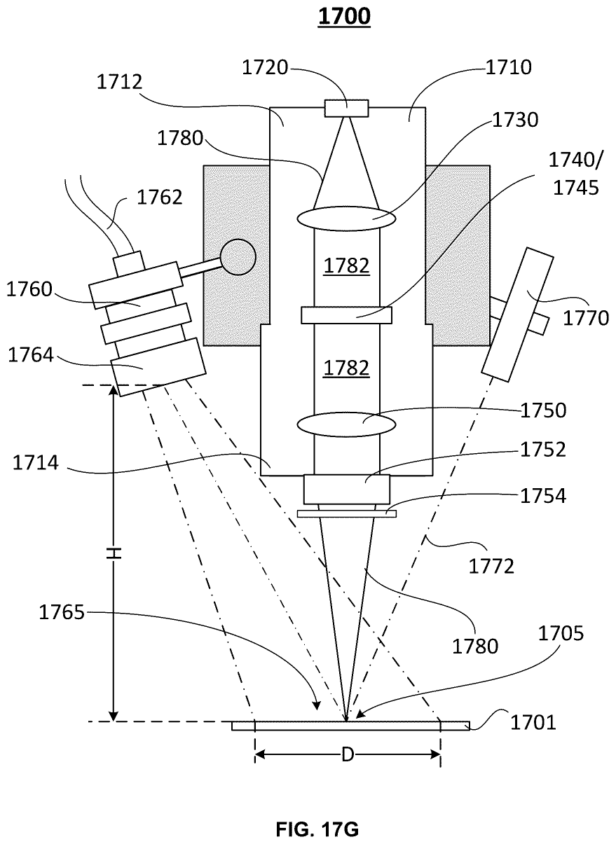

Referring to FIGS. 17A-17G, and particularly to FIG. 17G, the combined auto-fluorescence imaging and LSCI system are shown according to one embodiment of the invention. The system includes a light source for emitting a beam of light to illuminate a target of interest 1705; and an imaging head 1700 positioned over the target of interest 1705 for acquiring auto-fluorescence images and LSCI images of light from the illuminated target of interest 1705 responsive to the illumination.

In certain embodiments, the light source is, but not limited to, an infrared laser. The infrared laser is a diode laser emitting the beam of light at a wavelength of about 785 nm.

In the exemplary embodiment, the imaging head 1700 comprises a detector 1720 disposed in a top portion 1712 of the image head 1700 for individually acquiring the auto-fluorescence images and the LSCI images; and a first lens 1750 and a second lens 1730 positioned in an optical path 1782. The first lens 1750 is adapted for collecting the light from the illuminated target of interest 1705 in a surgical field, and the second lens is adapted focusing the collected light to the detector.

In certain embodiments, a ratio of a focal length of the first lens to that of the second lens is about 80/17. In one embodiment, the first lens is, but not limited to, a 400 mm lens, and the second lens is, but not limited to, an 85 mm lens. It should be appreciated that any set of lenses with a similar ratio of focal lengths can be utilized to practice the invention, for example, a 500 mm lens and a 100 mm lens. The choice of the 400 mm and 85 mm lenses was mainly to have a large working distance (400 mm) while keeping the imaging head relatively compact.

The imaging head 1700 also has a movable switching plate accommodating filters 1740 and an iris 1745, as shown in FIGS. 17E-17G, being located between the first lens 1750 and the second lens 1730. The movable switching plate operably moves between a first position and a second position. When the movable switching plate is in the first position, the filters 1740 are positioned in the optical path 1782 (FIG. 17E) and the detector operably acquires the auto-fluorescence images. When the movable switching plate is in the second position, the iris 1745 is positioned in the optical path (FIG. 17F) and the detector acquires the LSCI images. In one embodiment, the filters include, but are not limited to, a combination of an 808 nm long-pass filter and an 800 nm long-pass filter, and the iris comprises, but is not limited to, a 15 mm diameter iris. It should be appreciated that any long-pass or band-pass filters between the range of about 800 nm to about 830 nm can be utilized to practice the invention. In addition, for this configuration, any iris size less than 15 mm also works provided there is enough light. The 15 mm diameter of an iris is the limit to satisfy an equation regarding speckle size.

Furthermore, the imaging head 1700 includes a linear actuator (not shown) configured to move the movable switching plate between the first position and the second position.

In addition, the imaging head 1700 further comprises a focus tunable lens 1752 disposed in a bottom portion 1714 of the image head 1700 and positioned between the target of interest 1705 and the first lens 1750 in the optical path 1782 for focusing light 1780 from the illuminated target of interest 1705 in a surgical field.

Moreover, the imaging head 1700 comprises a first linear polarizer 1754 positioned in the optical path between the focus tunable lens 1752 and the target of interest 1705.

In certain embodiments, the detector 1720 comprises at least one camera. In one embodiment, the at least one camera comprises at least one charge-coupled device (CCD) camera and/or at least one complementary metal oxide semiconductor (CMOS) camera. In one embodiment, the at least one camera comprises at least one infrared camera and/or at least one camera near-infrared (NIR) camera.

In certain embodiments, the system further comprises at least one laser pointer 1770 arranged in relation to the detector 1720 such that its beam 1772 is co-localized with a center of the field of view of the detector 1720 at a distance. For example, two laser pointers, 532 nm and 650 nm, (output power <5 mW) attached on the sides of the imaging head 1700 guide a surgeon in positioning the imaging head 1700 so that the target of interest 1705 in roughly in the center of the field of view and in focus when imaging, as shown in FIG. 17G.

Also referring to FIG. 17G, the system also has a lens tube 1760 containing at least one lens arranged in relation to the target of interest 1705. The light source is optically coupled to the lens tube 1760 for illuminating a spot 1765 having a diameter D at a distance H on the target of interest 1705.

In addition, as shown in FIG. 17A, the system further comprises a controller (alternatively computer) configured to control operations of the imaging head for acquiring the auto-fluorescence and LSCI images of the illuminated target of interest, receiving the acquired auto-fluorescence and LSCI images from the detector, and processing the acquired auto-fluorescence and LSCI images to obtain speckle contrast images for the intraoperative assessment of parathyroid gland viability. In one embodiment, in the speckle contrast images, a perfused parathyroid gland has low speckle contrast, and a devascularized parathyroid gland has high speckle contrast.

In one embodiment, the system further comprises a display for displaying the speckle contrast images of the parathyroid gland in real-time, as shown in FIG. 17A.

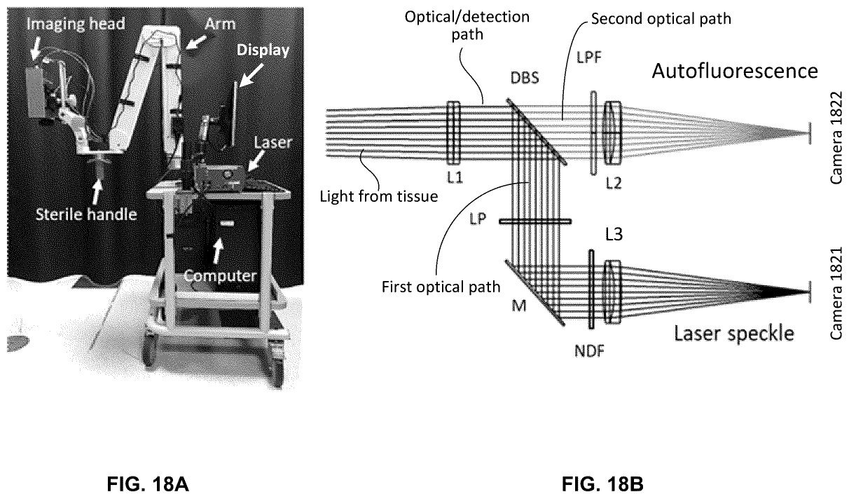

In another embodiment, as shown in FIG. 18A, the LSCI and auto-fluorescence imaging system is generally similar to LSCI and auto-fluorescence imaging system shown in FIG. 17A. Except that an alternative design of the imaging head, which includes two cameras 1821 and 1822 (FIG. 18B), instead of a single camera 1720 in the embodiment shown in FIGS. 17A-17G, and places a beamsplitter DBS in the optical/detection path to simultaneously collect fluorescence (by camera 1822) and laser speckle images (by camera 1821).

Specifically, as shown in FIG. 18B, the imaging head includes a beamsplitter DBS positioned between the first lens L1 and the second lens L2 in the optical path for reflecting and transmitting the collected light into a first path and a second path, respectively, and a third lens L3 positioned in the first path. The reflected light in the first path is focused by the third lens L3 to a first camera 1821 of the detector for acquiring the LSCI images, while the transmitted light in the second path is focused by the second lens L2 to a second camera 1822 of the detector for acquiring the auto-fluorescence images.

In addition, the imaging head further has a linear polarizer LP positioned between the beamsplitter DBS and the third lens L3 in the first path, and configured to have its axis of polarization oriented perpendicular to that on the illumination reducing specular reflections.

Furthermore, the imaging head further comprises a mirror M positioned between the linear polarizer LP and the third lens L3 in the first path for achieving compactness.

Also, the imaging head has an 808 nm long-pass filter LPF positioned between the beamsplitter DBS and the second lens L2 in the second path, and a neutral density filter NDF positioned between the beamsplitter DBS and the third lens L3 in the first path. The neutral density filter is configured to reduce the intensity of the laser light so it does not saturate the camera. In one embodiment, the neutral density filter is a 1.3 O.D. neutral density filter. Alternatively, if the laser is not so powerful it may not be needed, or a lower optical density may work, e.g., 0.5 O.D.

In another aspect of the invention, the method for intraoperative assessment of parathyroid gland viability of a living subject for guidance in a surgery includes providing a beam of light to illuminate a target of interest; acquiring auto-fluorescence images and laser speckle contrast imaging (LSCI) images of light from the illuminated target of interest responsive to the illumination; and processing the acquired auto-fluorescence and LSCI images for intraoperative guidance in a surgery.

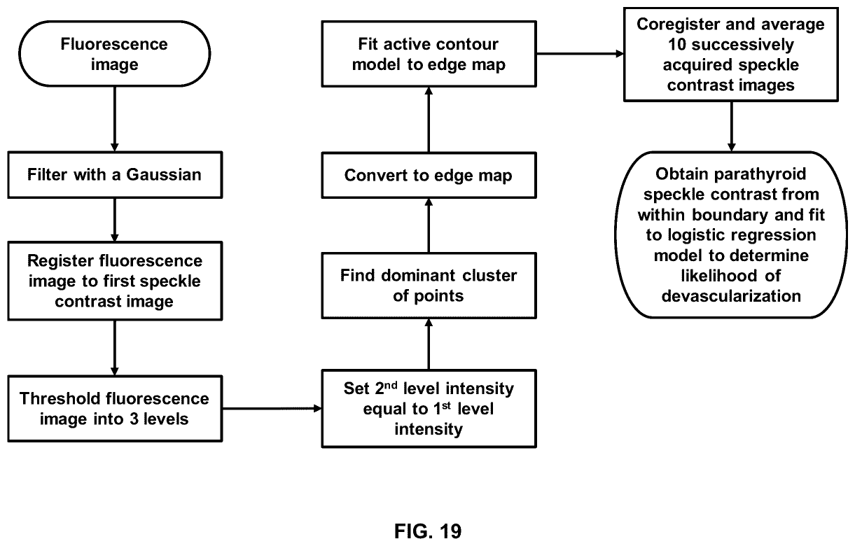

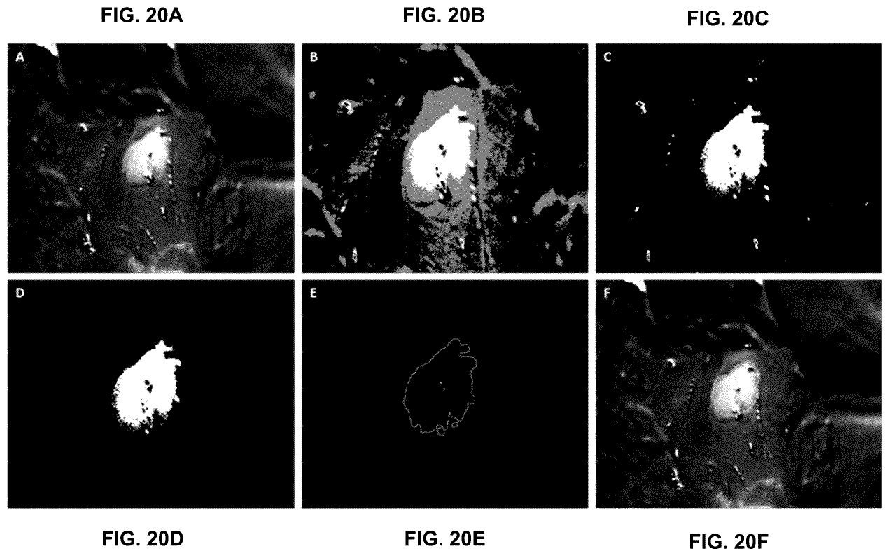

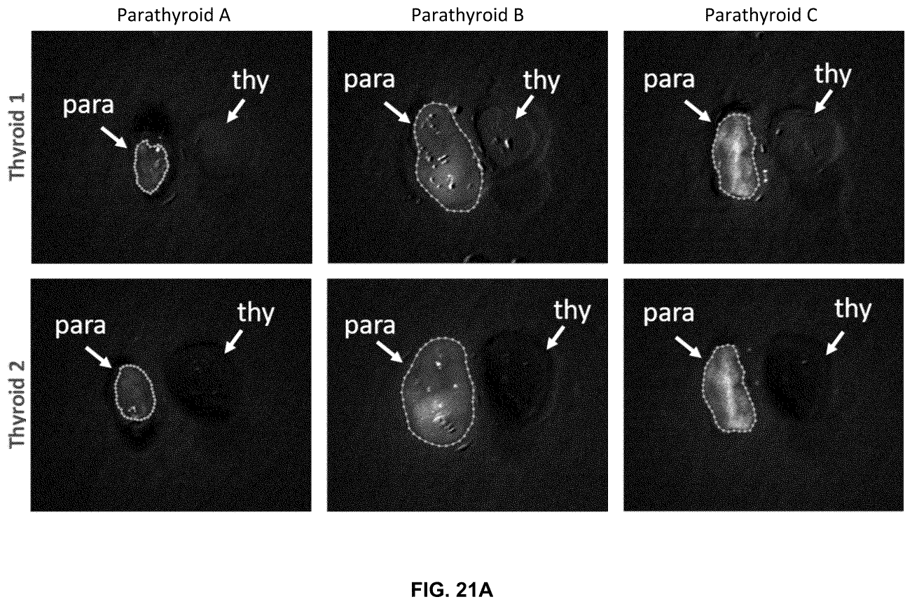

Referring to FIGS. 19 and 20A-20F, said processing the acquired auto-fluorescence and LSCI images comprises cropping a background-subtracted auto-fluorescence image (FIG. 20A) to remove first pixels on each edge; thresholding the cropped auto-fluorescence image into first, second and third intensity levels using a multiple thresholding scheme (FIG. 20B), wherein the first intensity level is corresponding to a low intensity background; setting the second intensity level equal to the low intensity background, resulting in an image (FIG. 20C) having a distinction between the parathyroid gland of interest and everything else; filtering the resulted image using a two-dimensional Gaussian to locate a dominant cluster of points, wherein the dominant cluster of points is corresponding to the parathyroid gland (FIG. 20D); converting the filtered image to an edge map (FIG. 20E); and fitting an active contour model to the edge map to obtain the contour demarcating the parathyroid gland (FIG. 20F).

Additionally, said processing the acquired auto-fluorescence and LSCI images comprises, prior to said the background-subtracted auto-fluorescence image, filtering an acquired auto-fluorescence image with a Gaussian profile; and registering the filtered auto-fluorescence image to a first speckle contrast image.

Further, said processing the acquired auto-fluorescence and LSCI images comprises imaging an irregular grid by two cameras to determine a rigid transformation that aligns the fields of the two cameras together, wherein one of the two cameras is adapted for acquiring the auto-fluorescence images and the other of two cameras is adapted for acquiring the LSCI images. In one embodiment, the rigid transformation is determined using an intensity-based image registration.

Moreover, said processing the acquired auto-fluorescence and LSCI images comprises after obtaining the contour, applying the rigid transformation to demarcate the parathyroid gland in the first speckle contrast image of the acquired series of speckle contrast images; registering the remaining subsequent speckle contrast images of the acquired series of speckle contrast images into the first speckle contrast image using a discrete Fourier transform registration that only accounts for translation; averaging the acquired series of speckle contrast images to obtain the average speckle contrast of the parathyroid area within the transformed contour so as to improve spatial resolution; and converting the value to a percent likelihood of parathyroid devascularization using a logistic regression model.

Also, the method includes for displaying the speckle contrast images of the parathyroid gland in real-time.

It should be noted that all or a part of the methods according to the embodiments of the invention is implemented by hardware or a program instructing relevant hardware.

### Imaging Systems

Embodiment 1: In this exemplary embodiment as shown in FIGS. 17A-17G, the imaging system is disposed on a cart (FIG. 17A) that can be wheeled into and out of the operating room. On the cart sits the computer that controls the instrument (home-built machine with six 3.7 GHz cores—Intel OEM Core i7-8700K), as well as a single mode 785 nm diode laser with 80 mW power output (Innovative Photonics Solutions, Monmouth Junction, NJ). Attached to the cart is an articulated arm (ICWUSA, Medford, OR) capable of extending about 4 feet from the edge of the cart, and having an attachment for a sterile handle to allow maneuvering by a surgeon. On the end of the arm is the imaging head 1700, see FIGS. 17B-17G, particularly FIG. 17G, which acquires both LSCI and fluorescence images by a detector 1720 disposed on an optical/detection path 1782. The laser source is coupled through a single mode fiber optic patch cable 1762 (Thorlabs, Newton, NJ) to a lens tube 1750 containing a 75 mm focal length lens (Edmund Optics, Barrington, NJ), illuminating a spot 1765 having a diameter D of about 45 mm at a distance H of about 450 mm. Also attached to this lens tube 1760 is a linear polarizer 1764 (Thorlabs, Newton, NJ)—together with another linear polarizer 154 attached to the front end of the detection path 1780, this enables reduction of specular reflections in images. The illumination has a Gaussian profile and the maximum power across the spot 1765 was measured to be 4 mW. A 532 nm and 650 nm laser pointer 1770 (output power less than 5 mW) attached on either side of a body portion 1710 of the imaging head 1700 (FIGS. 17D and 17G) guide the surgeon in positioning the system so that the tissue of interest in roughly in the center 1705 of the field of view at the object 1701 and in focus when imaging, as shown FIG. 17G.

The detection path 1782 basically includes two lenses: a 400 mm lens 1750 to collect light 1780 scattered from the tissue of interest responsive to the illumination from the imaging plane, and an 85 mm lens 1730 (Edmund Optics, Barrington, NJ) to focus this light 1780 onto the detector (camera sensor) 1720. The camera 1720 is a near-infrared optimized Basler acA1300-60gmNIR (Basler AG, Ahrensburg, Germany). The imaging head 1700 is designed to use one camera 1720 to acquire both fluorescence and laser speckle images. This is achieved by sliding either a combination 1740 of an 808 nm long-pass filter (Semrock, Rochester, NY) and an 800 nm long-pass filter (Midwest Optical Systems, Palatine, IL), or a 15 mm diameter iris 1745 into the detection path 1782 in-between the two lenses 1750 and 1730, where the filters 1740 are in place for fluorescence, and the iris 1745 for LSCI, respectively, (see FIGS. 17E-17G). Remotely switching the switching plate that accommodates the filters 1740 and iris 1745 (see FIGS. 17E-17F) between the filters 1740 and iris 1745 in the detection path 1782 is achieved using a linear actuator (Actuonix Motion Devices, Victoria, BC, Canada). In front of the 400 mm lens 1750 is a focus tunable lens 1752 (Optotune, Dietikon, Switzerland), the purpose of which is to enable remote image focusing, and attached to the front of the focus tunable lens 1752 is the another linear polarizer 1754.

This system is developed to guide surgeons performing thyroid and parathyroid surgeries, which allows a surgeon to objectively identify a parathyroid gland during surgery and assess its viability. Auto-fluorescence imaging helps identify the parathyroid, while LSCI helps assess its viability.

Embodiment 2: This exemplary embodiment is an alternative design of the imaging head that includes two cameras, instead of a single camera 1720 in the embodiment shown in FIGS. 17A-17G, and places a beam splitter in the optical/detection path to simultaneously collect fluorescence (by camera 1822) and laser speckle images (by camera 1821), as shown in FIGS. 18A-18B.

The LSCI and auto-fluorescence imaging system is developed in-house for the purpose of thyroid and parathyroid surgical guidance, as depicted in FIGS. 18A-18B, is generally similar to LSCI and auto-fluorescence imaging system shown in FIG. 17A. The device is constructed on a cart that can be wheeled into and out of the operating room. As shown in FIG. 18A, on the cart sits a computer that controls the instrument (home-built machine with six 3.7 GHz cores—Intel OEM Core i7-8700K), as well as a single mode 785 nm diode laser with 60 mW power output (Innovative Photonics Solutions, Monmouth Junction, NJ). Attached to the cart is an articulated arm (ICWUSA, Medford, OR) capable of extending about 4 feet from the edge of the cart, and having an attachment for a sterile handle to allow maneuvering by the surgeon. On the end of the arm is the imaging head that acquires both LSCI and auto-fluorescence images. The laser source is coupled through a single mode fiber optic patch cable (Thorlabs, Newton, NJ) to a lens tube attached on the exterior of the imaging head. This lens tube contains a 75 mm focal length lens (Edmund Optics, Barrington, NJ), illuminating a ˜30 mm diameter spot at a distance of 400 mm. Also attached to this lens tube is a linear polarizer (Thorlabs, Newton, NJ). The illumination has an approximately Gaussian profile and the maximum power across the spot was measured to be 4.8 mW. Two laser pointers, 532 nm and 650 nm, (output power <5 mW) attached on the sides of the imaging head guide the surgeon in positioning the device so that the tissue of interest in roughly in the center of the field of view and in focus when imaging.

Light scattered from the tissue is detected in the imaging head through one of two similar optical paths, depending on the wavelength. As shown in FIG. 18B, both paths share a 400 mm focal length lens L1 (Edmund Optics, Barrington, NJ) which collects the scattered light from the imaging plane and collimates it onto an 801 nm dichroic beamsplitter DBS (Semrock, Rochester, NY). The light scattered from the tissue (resulting in speckle images) reflects off the dichroic beamsplitter DBS and is focused by an 85 mm lens L3 (Edmund Optics, Barrington, NJ) onto a near-infrared-optimized camera 1822 (Basler AG, Ahrensburg, Germany). Between the dichroic beamsplitter DBS and the lens L3 is a second linear polarizer LP that has its axis of polarization oriented perpendicular to that on the illumination in order to reduce specular reflections. Also in front of the lens L3 is a 1.3 O.D. neutral density filter NDF (Thorlabs, Newton, NJ) to improve visualization of speckles on the sensitive camera 1822 (i.e. avoid saturation). The first optical path is folded to achieve compactness by inserting a silver mirror M (Thorlabs, Newton, NJ) after the polarizer LP. Modeling in Zemax 13 (Zemax, Kirkland, Washington) showed that this configuration results in a minimum detectable speckle size that is roughly twice the camera pixel size, which it has been shown that not matching this criterion leads to underestimation of speckle contrast when performing LSCI.

In the second optical path, the longer wavelength fluorescence is transmitted through the dichroic beamsplitter DBS and is further filtered by an 808 nm long-pass filter LPF (Semrock, Rochester, NY). It is then focused by another 85 mm lens L2 onto a second near-infrared-optimized camera 222. Lastly, there is a focus tunable lens (Optotune, Dietikon, Switzerland) attached to the front of the imaging head, before the 400 mm lens L1, which the purpose of focus tunable lens is to enable small remote adjustments in focus to help ensure images acquired in surgery are in focus. The field of view of the imaging system at the working distance of 400 mm is about 26×32 mm. The device is controlled with a custom program created using LabVIEW 2017 (National Instruments, Austin, TX).

### Intraoperative Imaging Procedure

Two patients undergoing parathyroidectomy at Vanderbilt University Medical Center were recruited under a larger study approved by the Institutional Review Board, and written informed consent was obtained prior to participation. Since transporting the device to and from the operating room (OR) over time could result in slight shifts in the positions of the two cameras, there is a need to know how offset they are for each case. Therefore, an irregular grid was imaged on a table off to one side of the OR before each case and the images obtained were used to determine the rigid transformation that aligns the fields of the two cameras together. This is done using intensity-based image registration and is accomplished with the click of a button on the LabVIEW program. Thereafter, once a parathyroid has been localized in a fluorescence image, this transformation can be applied to the identified contour to demarcate the parathyroid in the corresponding speckle contrast image and automatically determine viability. Intraoperative imaging followed one of two procedures depending on the health status of the gland being imaged. For a diseased gland, after locating and exposing the gland, the surgeon positions the device above the surgical field guided by the laser pointers. One set of images (a single fluorescence image and a series of speckle contrast images) is acquired at this point. Generating the fluorescence image involves first acquiring a background frame with the laser off, then acquiring a second image with the laser on and subtracting the former from the latter. Speckle contrast images are generated and displayed simultaneously in real time (about 24 fps) and the first 30 frames after acquiring the raw fluorescence image is saved. After acquiring the images, the surgeon then ligates the blood supply to the diseased parathyroid, which is a part of the standard procedure, and then a second set of images is acquired before excision of the gland. For a healthy gland, only one set of fluorescence and speckle contrast images is acquired and this could occur at any point during the surgery. The camera exposure times for imaging auto-fluorescence and speckle were 300 ms and 5 ms, respectively. During all imaging procedures, the room lights are left on. The surgeon's headlamp and the operating table lamp however have to be turned off or pointed away from the surgical field.

### Data Post-Processing

The algorithm developed to automatically segment the parathyroid is described herein according to one embodiment of the invention. Referring to FIGS. 19 and 20A-20B, first, the background-subtracted auto-fluorescence image (FIG. 20A) is cropped to remove the first 200 pixels on each edge (original image size is 1024×1280 pixels), and then thresholded into three levels using a multiple thresholding scheme based on Otsu's method (FIG. 20B). The reason for choosing three levels is that, while the parathyroid is generally the strongest auto-fluorescing tissue in the neck at this wavelength, other tissues such as the thyroid also emit significant fluorescence; choosing three levels allows separation of parathyroid, thyroid (and other less fluorescent tissues), and non-fluorescent background. After thresholding, the middle intensity level (thyroid) is set equal to the low intensity background (FIG. 20C). By this point there should ideally be a clear distinction between the parathyroid gland of interest and everything else, however noise due to specular reflections and imperfect thresholding may still contaminate the image. To reduce this, the next step is to filter the image using a two-dimensional Gaussian in order to locate the dominant cluster of points, the parathyroid gland (FIG. 20D). This filtered image can then be converted to an edge map (FIG. 20E), and an active contour model or “snake” can be fit to this edge map to obtain the contour demarcating the parathyroid gland (FIG. 20F). The force used to drive the active contour towards the parathyroid boundary is based on gradient vector flow. After obtaining the contour, the transformation that aligns the two cameras (determined from imaging the irregular grid) is applied to demarcate the parathyroid in the first of the acquired series of speckle contrast images. Nine subsequent speckle contrast images are registered to the first using a discrete Fourier transform registration that only accounts for translation—since these images are acquired at 24 fps, there is not much motion from frame to frame and a transformation that only relies on translation is sufficient to register them. These ten images are then averaged to improve spatial resolution and the average speckle contrast of the parathyroid (area within the transformed contour) is obtained. Finally, this value can be converted to a percent likelihood of parathyroid devascularization using a logistic regression model based on data accumulated in a previous study. The entire process described above takes about 5 seconds to run on the current computer.

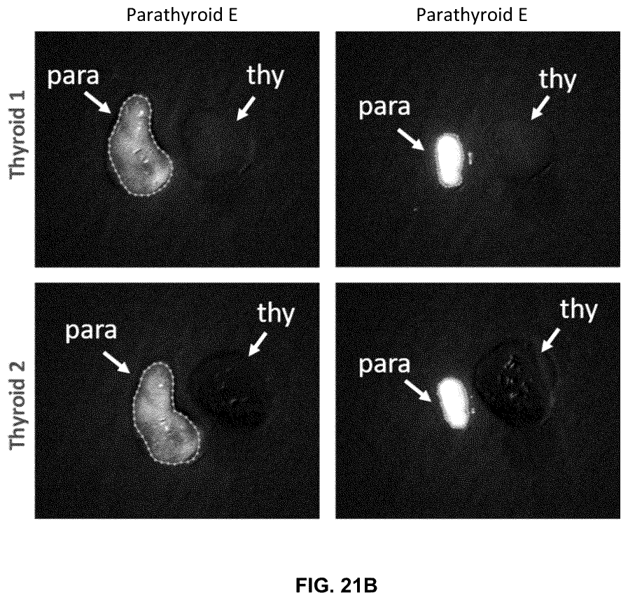

FIGS. 21A-21B show fluorescence images of parathyroid (para) and thyroid (thy) specimens in which the parathyroid has been successfully segmented out by the algorithm (indicated by the cyan contour). There are five parathyroids of different fluorescence intensities, each paired with two thyroid specimens.

### Example In Vivo Images

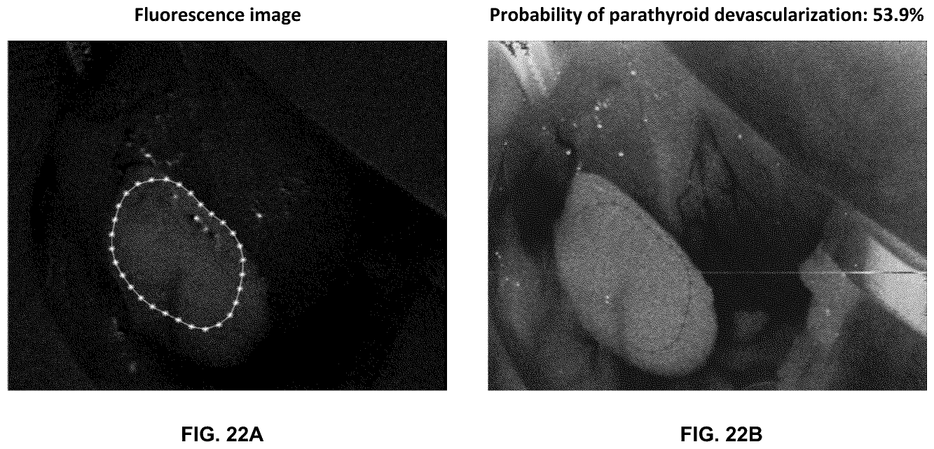

FIGS. 22A-22B show an in vivo example of a fluorescence image (FIG. 22A) and probability of parathyroid devascularization (FIG. 22B) according to one embodiment of the invention. Patient undergoing parathyroidectomy at Vanderbilt University Medical Center. Surgeon ligates blood supply to gland in preparation for removal; images taken after ligation. Parathyroid location used to extract viability information; speckle contrast converted to probability of devascularization using logistic regression model.

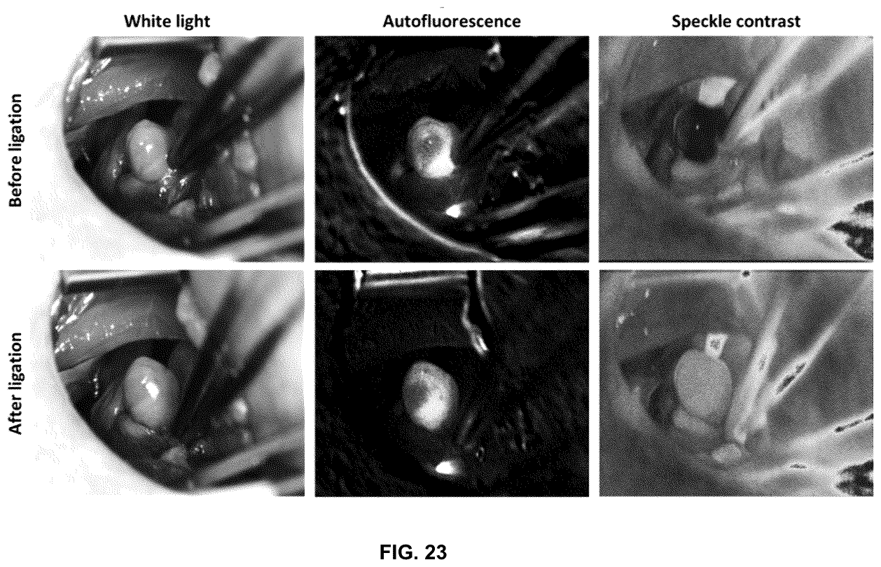

FIG. 23 shows a set of images obtained with the current iteration in a parathyroidectomy case, where images were obtained before and immediately after blood supply ligation. The parathyroid is clearly distinguished in the fluorescence images. As expected, the only observable difference between the before and after images is seen with LSCI where there is an increase in the parathyroid speckle contrast.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. Doherty, G. M. Thyroid and Parathyroid. in Oncology 992-1013 (2006).
[2]. Bliss, R. D., Gauger, P. G. & Delbridge, L. W. Surgeon's approach to the thyroid gland: surgical anatomy and the importance of technique. World journal of surgery 24, 891-897 (2000).
[3]. Miller, F. R. Surgical anatomy of the thyroid and parathyroid glands. Otolaryngologic clinics of North America 36, 1-7, vii (2003).
[4]. ATA. Thyroid Surgery. (American Thyroid Association, 2005).
[5]. Prosst, R. L., Gahlen, J., Schnuelle, P., Post, S. & Willeke, F. Fluorescence-guided minimally invasive parathyroidectomy: a novel surgical therapy for secondary hyperparathyroidism. Am J Kidney Dis 48, 327-331 (2006).
[6]. Frilling, A. & Weber, F. Complications in Thyroid and Parathyroid Surgery. in Surgery of the Thyroid and Parathyroid Glands 217-224 (2007).
[7]. Ahuja, A. T., et al. Imaging for primary hyperparathyroidism—what beginners should know. Clinical radiology 59, 967-976 (2004).
[8]. Fakhran, S., Branstetter, B. F. t. & Pryma, D. A. Parathyroid imaging. Neuroimaging clinics of North America 18, 537-549, ix (2008).
[9]. Ramanuj am, N., et al. Spectroscopic diagnosis of cervical intraepithelial neoplasia (CIN) in vivo using laser-induced fluorescence spectra at multiple excitation wavelengths. Lasers in surgery and medicine 19, 63-74 (1996).
[10]. Lakowicz, J. R. Fluorophores. in Principles of Fluorescence Spectroscopy 63-95 (2006).
[11]. Kim, L. a. A. M. Hyperparathyroidism. (ed. Daniel Einhorn, F. T., Don S Schalch, Mark Cooper and George T Griffing) (Webmd, 2008).
[12]. Lakowicz, J. R. Introduction to Fluorescence. in Principles of Fluorescence Spectroscopy 1-26 (2006).
[13]. Monici M., Cell and tissue autofluorescence research and diagnostic applications. Biotechnol Annu Rev. 11:227-56 (2005).
[14]. American Thyroid Association. General Information/Press Room. (2017). at <http://www.thyroid.org/media-main/about-hypothyroidism/>
[15]. Sun, G. H., DeMonner, S. & Davis, M. M. Epidemiological and Economic Trends in Inpatient and Outpatient Thyroidectomy in the United States, 1996-2006. Thyroid 23, 727-733 (2013).
[16]. Rafferty, M. A. et al. Completion Thyroidectomy Versus Total Thyroidectomy: Is There a Difference in Complication Rates? An Analysis of 350 Patients. J. Am. Coll. Surg. 205, 602-607 (2007).
[17]. Schwartz, A. E. & Friedman, E. W. Preservation of the parathyroid glands in total thyroidectomy. Surg. Gynecol. Obstet. 165, 327-32 (1987).
[18]. Ritter, K., Elfenbein, D., Schneider, D. F., Chen, H. & Sippel, R. S. Hypoparathyroidism after total thyroidectomy: incidence and resolution. J. Surg. Res. 197, 348-53 (2015).
[19]. Demeester-Mirkine, N., Hooghe, L., Van Geertruyden, J. & De Maertelaer, V. Hypocalcemia after thyroidectomy. Arch. Surg. 127, 854-8 (1992).
[20]. Pattou, F. et al. Hypocalcemia following Thyroid Surgery: Incidence and Prediction of Outcome. World J. Surg 22, 718-724 (1998).
[21]. McHenry, C. R., Speroff, T., Wentworth, D. & Murphy, T. Risk factors for postthyroidectomy hypocalcemia. Surgery 116, 641-7-8 (1994).
[22]. Zahedi Niaki, N. et al. The Cost of Prolonged Hospitalization due to Postthyroidectomy Hypocalcemia: A Case-Control Study. Adv. Endocrinol. 2014, 1-4 (2014).
[23]. Naveh-Many, T. Molecular Biology of the Parathyroid. (Kluwer Academic/Plenum Publishers, 2005). at <http://web.b.ebscohost.com.proxy.library.vanderbilt.edu/ehost/ebookviewer/ebook/bmx1 YmtfXzEyNTkwNF9fQU41?sid=0747ff07-a77e-43ea-9efe-aa979596f166@sessionmgr2&vid=0&format=EB&lpid=lp_x&rid=0>
[24]. Mohebati, A. & Shaha, A. R. Anatomy of thyroid and parathyroid glands and neurovascular relations. Clin. Anat. 25, 19-31 (2012).
[25]. Lo, C. Y. et al. Parathyroid Autotransplantation During Thyroidectomy. Arch. Surg. 136, 1381 (2001).
[26]. Sierra, M. et al. Prospective biochemical and scintigraphic evaluation of autografted normal parathyroid glands in patients undergoing thyroid operations. Surgery 124, 1005-(1998).
[27]. Kuriloff, D. B. & Kizhner, V. Parathyroid gland preservation and selective autotransplantation utilizing topical lidocaine in total thyroidectomy. Laryngoscope 120, 1342-1344 (2010).
[28]. Ezzat, W. et al. Intraoperative parathyroid hormone as an indicator for parathyroid gland preservation in thyroid surgery. Swiss Med. Wkly. (2011). doi:10.4414/smw.2011.13299
[29]. Fortuny, J. V. et al. Parathyroid gland angiography with indocyanine green fluorescence to predict parathyroid function after thyroid surgery. Br. J. Surg. 103, 537-543 (2016).
[30]. Boas, D. A. & Dunn, A. K. Laser speckle contrast imaging in biomedical optics. J. Biomed. Opt. 15, 11109 (2010).
[31]. Choi, B., Kang, N. M. & Nelson, J. S. Laser speckle imaging for monitoring blood flow dynamics in the in vivo rodent dorsal skin fold model. Microvasc. Res. 68, 143-6 (2004).
[32]. Iredahl, F., Löfberg, A., Sjöberg, F., Farnebo, S. & Tesselaar, E. Non-Invasive Measurement of Skin Microvascular Response during Pharmacological and Physiological Provocations. PLoS One 10, e0133760 (2015).
[33]. Dunn, A. K., Bolay, H., Moskowitz, M. A. & Boas, D. A. Dynamic Imaging of Cerebral Blood Flow Using Laser Speckle. J. Cereb. Blood Flow Metab. 21, 195-201 (2001).
[34]. Huang, Y.-C., Ringold, T. L., Nelson, J. S. & Choi, B. Noninvasive blood flow imaging for real-time feedback during laser therapy of port wine stain birthmarks. Lasers Surg. Med. 40, 167-173 (2008).
[35]. Mirdell, R., Iredahl, F., Sj?berg, F., Farnebo, S. & Tesselaar, E. Microvascular blood flow in scalds in children and its relation to duration of wound healing: A study using laser speckle contrast imaging. Burns 42, 648-654 (2016).
[36]. Richards, L. M. et al. Intraoperative multi-exposure speckle imaging of cerebral blood flow. J. Cereb. Blood Flow Metab. 0271678X1668698 (2017). doi:10.1177/0271678X16686987
[37]. Sexton, K. J., Zhao, Y., Davis, S. C., Jiang, S. & Pogue, B. W. Optimization of fluorescent imaging in the operating room through pulsed acquisition and gating to ambient background cycling. Biomed. Opt. Express 8, 2635 (2017).
[38]. Kirkpatrick, S. J., Duncan, D. D. & Wells-Gray, E. M. Detrimental effects of speckle-pixel size matching in laser speckle contrast imaging. Opt. Lett. 33, 2886 (2008).
[39]. Briers, D. et al. Laser speckle contrast imaging: theoretical and practical limitations. J. Biomed. Opt. 18, 66018 (2013).
[40]. Kirkpatrick, S. J., Duncan, D. D. & Wells-Gray, E. M. Detrimental effects of speckle-pixel size matching in laser speckle contrast imaging. Opt. Lett. 33, 2886 (2008).
[41]. Reddi, S. S., Rudin, S. F. & Keshavan, H. R. An optimal multiple threshold scheme for image segmentation. IEEE Trans. Syst. Man. Cybern. SMC-14, 661-665 (1984).
[42]. Xu, C. & Prince, J. L. Snakes, shapes, and gradient vector flow. IEEE Trans. Image Process. 7, 359-369 (1998).
[43]. Guizar-Sicairos, M., Thurman, S. T. & Fienup, J. R. Efficient subpixel image registration algorithms. Opt. Lett. 33, 156 (2008).
[44]. Mannoh, E. A., Thomas, G., Solorzano, C. C. & Mahadevan-Jansen, A. Intraoperative Assessment of Parathyroid Viability using Laser Speckle Contrast Imaging. Sci. Rep. 7, 14798 (2017).
[45]. Sun, G. H., DeMonner, S. & Davis, M. M. Epidemiological and Economic Trends in Inpatient and Outpatient Thyroidectomy in the United States, 1996-2006. Thyroid 23, 727-733 (2013).
[46]. Pattou, F., Combemale, F., Fabre, S., Carnaille, B., Decoulx, M., Wemeau, J., Radacot, A. & Proye, C. Hypocalcemia following Thyroid Surgery: Incidence and Prediction of Outcome. World J Surgery 22, 718-724 (1998).
[47]. Paras, C., Keller, M., White, L., Phay, J. & Mahadevan-Jansen, A. Near-infrared autofluorescence for the detection of parathyroid glands. J. Biomed. Opt. 16, 067012 (2011).

What is claimed is:

1. A system facilitating differentiation between parathyroid tissue and thyroid tissue of a subject, comprising:
a light source for illuminating tissue in a target region in the neck area of the subject;
a light delivery device that is optically coupled with the light source for delivering a beam of light from the light source to the tissue;
a collection device for collecting optical signals emitted from the tissue responsive to the beam of light; and
a detector for detecting the intensities of the optical signals emitted from the tissue, wherein differences in the detected intensities enable a user to differentiate between parathyroid tissue and thyroid tissue.

2. The system according to claim 1, wherein the beam of light has a wavelength in the near-infrared range.

3. The system according to claim 1, wherein the collection device is configured to collect optical auto-fluorescence signals.

4. The system according to claim 1, further comprising a display in communication with the detector for displaying the intensities of the optical signals emitted from the tissue.

5. The system according to claim 1, further comprising a filter for filtering excess ambient light to ensure desirable optical signals to be detected.

6. The system according to claim 5, wherein the light source is a near-infrared light source.

7. The system according to claim 5, wherein the collection device is configured to collect optical auto-fluorescence signals.

8. The system according to claim 5, further comprising a display in communication with the detector for displaying the intensities of the optical signals emitted from the tissue.
